(19) United States
(12) Patent Application Publication
(10) Pub. No.: US 2012/0058936 A1
(43) Pub. Date: Mar. 8, 2012

(54) COMPOSITIONS AND METHODS FOR ELIMINATION OF GRAM NEGATIVE BACTERIA

(75) Inventors: Antoine Andremont, Malakoff (FR); Jean De Gunzburg, Londres (GB); Francois Lescure, Pechbusque (FR)

(73) Assignees: ASSISTANCE PUBLIQUE - HOPITAUX DE PARIS, Paris (FR); DA VOLTERRA, Paris (FR); UNIVERSITE PARIS DIDEROT - PARIS 7, Paris (FR)

(21) Appl. No.: 13/256,220

(22) PCT Filed: Mar. 12, 2010
(86) PCT No.: PCT/EP10/53231

§ 371 Date: Nov. 10, 2011

Related U.S. Application Data
(60) Provisional application No. 61/160,162, filed on Mar. 13, 2009.

(51) Int. Cl. A61K 38/12; A61P 31/04; A61P 37/06; A61P 37/08; A61P 31/12; A61K 38/02; A61P 29/00

(52) U.S. Cl. 514/2.8

(57) ABSTRACT

Oral drug delivery formulations which specifically administer antibacterial agents to the ileum, caecum, and/or the colon, without significant administration elsewhere in the gastrointestinal tract, are disclosed. The formulations include, as actives, a combination of a macrolide or aminoglysoside, or quinolone antibacterial and an anti-Gram-negative lipopeptide (polymyxin) antibacterial agent or other peptide antibacterials effective against Gram-negative bacteria. The formulations can be used to treat infections or unwanted colonization in the colon, and to provide effective decontamination of the colonic flora from unwanted or potentially pathogenic bacteria.

### FIELD OF THE INVENTION

The present invention is in the area of oral drug delivery systems to administer antibacterial agents to the ileum, caecum, and/or the colon. More specifically, the present invention relates to the oral administration, and delivery to the distal ileum, caecum, and/or colon, of a combination of a macrolide, quino lone, or aminoglycoside antibacterial and a lipopeptide antibacterial, such as a polymyxinantibacterial, to treat infections or unwanted colonization in the colon, and for the effective elimination from colonic flora of potential pathogenic bacteria.

### BACKGROUND OF THE INVENTION

There are typically many types of bacteria in the colon, including beneficial bacteria and bad (pathogenic) bacteria. There are a number of colonic bacterial infections that can result in significant mortality and/or morbidity. Examples of these include infections caused by certain strains of E. coli.

In a number of instances the colon is colonized by antibiotic-resistant and potentially pathogenic bacteria, such as Enterobacteria or other Gram-negative bacteria such as Pseudomonas, Acinetobacter or other non fermentative Gram-negative bacteria. Orally administered antibacterials have been used to eliminate these bacteria. This practice is referred to “selective digestive decontamination” or SDD. (Selective decontamination of the digestive tract. de Smet A M, Bonten M J. Curr Opin Infect Dis. 2008 April; 21(2):179-83).

The intestinal colonic flora is recognized as a major system in the dynamic of emergence and spread of bacterial resistance to antibacterial agents for 5 main reasons.

First, it is composed of several hundreds of bacterial species in dense populations which can host resistant bacteria among them.

Second, bacteria from the commensal flora which are susceptible to antibacterials can become resistant after lateral transfer of resistance gene(s) from resistant bacteria from the environment penetrating the intestinal tract for example with food, or from other bacteria already present in the gastro-intestinal tract.

Third, bacteria from the commensal flora which are susceptible to antibacterials can become resistant after selection of naturally arising resistant mutants during antibacterial treatments.

Fourth, the resistant bacteria from the commensal flora, whatever their origin, disseminate in the environment when eliminated with the feces.

Fifth, the resistant bacteria from the commensal flora can further transfer their genes of resistance to other bacteria, either within the intestinal tract or in the environment to other bacterial species which can be either virulent/pathogenic for humans and/or animals, or non virulent, thus further increasing the burden and extend of bacterial resistance to antibacterials.

These events may have two types of deleterious consequences. First, patients whose colon is colonized by resistant bacteria are at risk of developing infections caused by these resistant bacteria, should they encounter circumstances which favor such occurrence. Examples of such circumstances are the occurrence of urinary tract infections, a very common infection in women, or the occurrence of systemic generalized infections in immunocompromised patients, or in those undergoing surgery, those hospitalized in intensive care units, those in whom prosthetic materials are implanted, and the like. Second, the colonized subjects can disseminate resistant bacteria in the environment or to other subjects, both in the hospital setting and in the community.

Therefore it would be advantageous for the clinician to have at hand a composition that eliminates such resistant bacteria from the colonic flora of the colonized patients without causing any substantial side effect to the subjects. Such side effects are well-known with actual treatments, and it would be advantageous that such treatment be more respectful to the intestinal tract, limiting or avoiding local or general side effects.

More specifically, these side effects generally include, at the intestinal level, side effects such as nausea, vomiting, gastric discomfort, diarrhea, constipation and so forth. An additional side effect is the selection of bacteria resistant to the drug used, and colonization by such bacteria with all of the possible consequences described above for the initially-colonizing bacteria. Of particular concern is the potential occurrence of infections caused by such bacteria, which are difficult to treat because they are resistant to many antibacterial agents, such as the initially colonizing bacteria, but also to the agents used for their elimination, and therefore accessible to only a very limited number of treatments.

It would be advantageous also to limit or avoid significant absorption of the antibacterials used for SDD at the systemic level, while targeting local treatment, to avoid known side effects such as secondary effects and toxicity, which often occur after systemic absorption of the antibacterial agent.

For all of the above reasons, an ideal product designed for SDD would have the following properties.

First, it should be easily administrable to the colonized subjects, ideally by oral administration. Second, it should demonstrate high efficacy in eliminating the intestinal colonizing target resistant strains, and it should minimize the emergence of resistance among target organisms. Third, it should limit the systemic absorption of antibacterials by delivering such products to the late ileum or colon. Fourth, it should demonstrate significant reduction of the side effects described above, local or general.

The present invention provides such a composition, and methods for eliminating Gram-negative resistant bacteria from the intestinal tract of colonized subjects using the composition.

As a general statement, the circumstances in which the colon can be colonized by resistant and potentially pathogenic bacteria, such as Enterobacteria or other Gram-negative bacteria such as Pseudomonas, Acinetobacter or other non fermentative Gram-negative bacteria are numerous and can include colonization after antibacterial treatment for whatever cause, and contamination from the environment, food or other external sources.

The consequences of such colonization of the colon are numerous. The major one being the occurrence of infections in the patients or in the animal that is colonized. This can occur with particular high frequency when treating immunocompromized patients. This can also occur during any medical treatment that would decrease resistance to infection. Examples of such medical treatments can be during surgical procedures such as the ones performed for the implantation into the patients of organs (artificial or of human/animal origin) prosthesis, catheters, cardiac valves and so forth.

Infections can also occur in non immunocompromised patients when the source of the infection is the intestinal Gram-negative microflora as, for example, in urinary tract infections (J Clin Nurs. 2002 September; 11(5):568-74. Pathogenesis of urinary tract infections: a review. Moore K N, Day R A, Albers M.)

The consequences of colonization of the intestinal tract by resistant and potentially pathogenic bacteria, such as Enterobacteria or other Gram-negative bacteria such as Pseudomonas, Acinetobacter or other non fermentative Gram-negative bacteria can also be the dissemination of such bacteria in the environment (The role of the intestinal tract as a reservoir and source for transmission of nosocomial pathogens. Donskey C J. Clin Infect Dis. 2004 Jul. 15; 39(2):219-26; Antibacterial regimens and intestinal colonization with antibacterial-resistant Gram-negative bacilli. Donskey C J. Clin Infect Dis. 2006 Sep. 1; 43 Suppl 2:S62-9) or their transfer from one colonized subject to an healthy subject resulting in outbreaks of nosocomial infections (Outbreak of multidrug-resistant Acinetobacter baumannii in a Belgian university hospital after transfer of patients from Greece. Wybo I, Blommaert L, De Beer T, Soetens O, De Regt J, Lacor P, Piérard D, Lauwers S. J Hosp Infect. 2007 December; 67(4):374-80).

It would be a major clinical breakthrough to specifically deliver antibacterial agents to the colon in at least two different clinical conditions: first to treat established colonic infections, and second, to eliminate asymptomatic colonization by unwanted microbes (resistant and/or potentially pathogenic ones) as a measure aimed at preventing the spread of the unwanted microbes in the environment (hospital, nursing/special care home, or regular home) and at preventing the occurrence of subsequent infections in the colonized hosts.

In order to do so one would need to deliver to the colon, efficient concentrations of active agents that, besides the desired effect, do not promote bacterial resistance, and/or are selective for harmful (pathogenic) or unwanted microbes or bacteria over helpful bacteria, which ideally should remain largely unaffected.

In addition one would need to prevent any other action or side-effect of the antibacterial agents delivered to the colon including any pharmacological effect on the physiology of the intestinal tract such as prokinetic effects on the stomach or the small intestine resulting in accelerated transit or any systemic toxicity and side effects (renal, hepatic or others) which can follow the absorption of the antibacterial by the intestinal tract and the dissemination of the antibacterial in other parts of the body than the colon.

Several examples of the clinical advantages of specific and targeted delivery of antibacterials to the colon are listed below:

Concerning colonization by unwanted microbes and their elimination, a medical practice referred to as “selective digestive decontamination” (SDD) has, as stated above, been described and used with this goal for over 30 years. It aims at eliminating commensal and/or potentially pathogenic microorganisms (such as for example enterobacteria, pseudomonas, enterococci) from the colon of very sick patients at high risk of infection (such as intensive care, or haemato-onco logy patients, for example) before they develop an actual infection. However, SDD has never gained general acceptance in spite of its most probably favorable effect to prevent the occurrence of Gram-negative infections in these patients at very high risk (Impact of SDD of the digestive tract on carriage and infection due to Gram-negative and Gram-positive bacteria: a systematic review of randomized controlled trials. Anaesth Intensive Care. 2008 May; 36(3):324-38. Silvestri L, van Saene H K, Casarin A, Berlot G, Gullo A.).

This lack of confidence of the medical community in the effectiveness of SDD is due to several factors:

First, the decontamination is without effect on the occurrence of Gram-positive infections, because it currently relies on anti-Gram-negative antibacterials. This reduces the overall interest of the practice in the very high risk patients because they are also at high risk of Gram positive infections. In a pivotal study the authors showed that the reduction in Gram-negative infections observed when SDD was used in ICU patients under mechanical ventilation was associated with an increased in Gram positive infections, thus annulling all beneficial effect of SDD.

Second, the antibacterials used for SDD belong to the same antibacterial families which are also used for treating infected patients (for instance, aminoglycosides or fluoroquinolones or colistin). This is associated with the fear that large uses of these agents for selective decontamination will ultimately select resistant bacteria, which will result in infections that are untreatable by most of the currently-available antibacterials.

Another possible use of SDD can also be found in farm animals. Indeed, bovines and ovines are often asymptomatically colonized by specific types of Escherichia coli strains, namely Shiga-toxin Escherichia coli (or STEC), also called Verotoxin Escherichia coli (or VETEC) (Treatment and prevention of enterohemorrhagic Escherichia coli infection and hemolytic uremic syndrome. Expert Rev Anti Infect Ther. 2007 August; 5(4):653-63. Goldwater P N.). These strains can contaminate foodstuff, including meat during slaughtering, milk during the milking process, and fruits and vegetables to be eaten raw after the use of contaminated water for irrigation or animal waste for manure.

Cases have also been observed after recreational activities in water contaminated by animal waste. Ingestion of only a few number of STEC (10-100 cells) can cause bloody diarrhea in humans, particularly in young children. In addition approximately 5% of the subjects that develop such diarrhea will suffer in the following weeks of a much more severe disease called hemolytic and uremic syndrome (or SHU). SHU is a severe disease often requiring hospitalization and extra-renal epuration. The death rate is around 5%. There is no current means of proven efficacy for decreasing STEC colonization in the livestock (Fairbrother J M, Nadeau E Escherichia coli: on-farm contamination of animals, Rev Sci Tech. 2006 August; 25(2):555-69). However, targeting effective antibacterials to the colon of colonized animals could achieve this goal.

SDD has also been advocated to help control outbreaks of antibacterial resistant Gram-negative infections in hospitals (Brun-Buisson C, Legrand P, Rauss A, Richard C, Montravers F, Besbes M, Meakins J L, Soussy C J, Lemaire F, Intestinal decontamination for control of nosocomial multiresistant Gram-negative bacilli. Study of an outbreak in an intensive care unit. Ann Intern Med. 1989 Jun. 1; 110(11):873-81.) This use was advocated mostly against outbreaks of nosocomial infections caused by enterobacteria (mostly Klebsiella) resistant to third generation cephalosporins by secretion of an extended spectrum beta-lactamase (ESBL) derived from the TEM or SHV beta-lactamase families. Such bacteria were mostly observed in the hospital setting and very rarely in the community. However, this type of ESBL and the associated outbreaks have currently mostly disappeared from the hospitals of many countries when the use of hand hygiene with alcoholic solutions and contact precautions have been implemented to a large scale in hospitals to prevent bacterial cross transmission between patients. However, a new type of ESBL, called CTX-M, which have a strikingly different epidemiological pattern of emergence and diffusion, are currently prevalent in hospitals (Pitout J D, Laupland K B. Extended-spectrum beta-lactamase-producing Enterobacteriaceae: an emerging public-health concern. Lancet Infect Dis. 2008 March; 8(3):159-66). CTX-M enterobacteria are not only resistant to third generation cephalosporins, just as ESBL ones, but they are widely present both in the community where they seems to emerge first and in the hospitals which appear to be invaded from the outside by virtue of community patients already colonized when they are admitted. In addition, CTX-M are most often carried by enterobacterial species (such as Escherichia coli) much better fitted to the intestinal tract ecosystem than those carrying previously known ESBL. Thus, they apparently have very little tendency to be spontaneously eliminated. Because of these combined facts, colonization by CTX-M enterobacteria is considered to be a major threat for the sake of antibacterial treatments in the coming years. In addition a number of enterobacteria have now been described that carry carbapenemase enzymes which can cause an even more important threat because they are not sensitive to any of the beta lactam antibacterials currently available (Carbapenemases: molecular diversity and clinical consequences. Poirel L, Pitout J D, Nordmann P. Future Microbiol. 2007 October; 2(5):501-12.).

A possible new strategy, indeed rather different from that of the classical use of SDD, would be to eliminate Gram-negative resistant bacteria from the colon of the patients colonized, upon admission to the hospitals, for instance after screening, using specific antibacterial agents targeted to the colon.

Then, it would be advantageous to provide compositions and methods for delivering such agents to the late ileum or colon in their active form after oral administration. The present invention provides such compositions and methods.

### SUMMARY OF THE INVENTION

Compositions and methods for providing elimination of Gram-negative bacteria in the colon, and/or treating a Gram-negative bacterial infection in the colon, are disclosed. The compositions comprise a combination of an aminoglycoside, macrolide or quinolone antibacterial with an anti-Gram-negative lipopeptide antibacterial, (such as a polymyxin-type antibacterial) or other peptide antibacterial effective against Gram-negative bacteria.

The compositions are specifically designed to locally deliver the active drug to the late ileum or colon, while limiting systemic absorption in the upper tract to limit or avoid any unwanted side effects. Methods for preparing the drug delivery systems and for providing elimination of Gram-negative bacteria in the colon using such drug delivery systems, are also disclosed.

Certain polymyxin antibacterials, such as colistin (also called colimycin or polymyxin E), are toxic if administered systemically (i.e. by intravenous injection), but have been used orally for intestinal disinfection for many years (Antibacterial agents edited by André Bryskier Published by ASM press, Washington, in 2005, Chap 30).

Macrolide antibacterials treatments often result in the development of bacterial resistance to macrolides, but such bacteria can be treated with other antibacterials, for example, quinolone antibacterials. In contrast, if a quinolone were used in combination with the lipopeptide antibacterial, and a quino lone-resistant bacteria were produced as a result, the macrolide antibacterial would likely not be able to treat such bacterial infection. For this reason, combinations of macrolide antibacterials with the lipopeptide antibacterials are preferred over combinations of quinolone antibacterials with the lipopeptide antibacterials.

Combinations of aminoglycoside antibacterials with the lipopeptide antibacterials are also preferred.

That said, by using a specific combination of both
a polymyxin, or any other anti Gram-negative lipopolypeptide antibacterial or other peptide antibacterial effective against Gram-negative bacteria), and
a macrolide or quinolone or aminoglycoside antibacterial

for elimination of resistant Gram-negative bacteria from the colon, the problem of bacterial resistance developed against one agent is minimized.

In one aspect of these embodiments, the agents are administered orally, but are specifically delivered and released at the colon site. As used herein, colonic delivery includes delivery to the distal ileum/caecum/colon, although colonic delivery is preferred.

There are many means known for delivering a drug to the colon. Enteric coating that protect the composition against gastric attack in the upper intestinal tract and release the active drug in the lower intestinal tract have been developed for years (Singh et al., Modified-Release solid formulations for colonic delivery, in Recent patents on drug delivery & formulation, 2007, 1, 53-63; Rubinstein et al., Colonic drug delivery; in Drug discovery today: technologies, 2005 Vol. 2, No. 1, 33-37)

The active agents can be formulated in capsules, tablets or any acceptable pharmaceutical composition, and are ideally designed to specifically release the active agents in a programmed manner at a specific site of the intestinal tract, such as but not limited to the distal ileum or colon. The programmed delivery prevents the agents from being degraded in the stomach after oral absorption, and permits them to be released into the lower part of the small intestine, i.e. the ileum, and the colon. In one embodiment, the compositions allow the formulated active agents to recover their maximum antibacterial capabilities when they reach the desired part of the intestinal tract.

In one embodiment, the combined antibacterials can be administered as two different products or combined in the same formulation at a given efficient dose. For example, the antibacterials can be combined in a single formulation for ileal/caecal/colonic delivery. In another illustrative embodiment, the antibacterials are formulated independently. In this embodiment, one may then include the two independent formulations in one single capsule or in distinct capsules (these distinct capsules can be associated in the same blister or separated in different blister in the end-user product). In another illustrative embodiment, the lipopeptide (or peptide) antibacterial is formulated for delivery in the late ileum, caecum or colon and the macrolide, aminoglycoside or quinolone antibacterial is not formulated for delayed release. In this latter embodiment, both antibacterials may be included in the same product (for example in the same capsule, or tablet) or in different products (for example in different tablets or capsules, these different products being or not in the same blister).

It might be desirable to allow flexibility in the doses that the clinician will be able to administer and consider two different products that will be designed for colonic delivery of each antibacterial. However a combined pharmaceutical dosage form that contains both antibacterials at the given efficient dose might be suitable for most cases. Formulation is preferentially designed to
(i) Prevent any systemic absorption of the antibacterial, and then eliminate any possible systemic toxicity or side effect,
(ii) Prevent any effect on the upper intestinal tract, and
(iii) Achieve luminal concentrations in the late ileum, the caecum and the colon which are high enough to kill the target organisms, which are the unwanted Gram-negative bacteria, and also prevent emergence of resistance among these bacterial populations.

Thus the invention relates to a composition comprising a drug delivery system that is orally administered, and delivers specifically to the late ileum, caecum, or colon, and substantially avoids delivery to other areas of the gastrointestinal tract, wherein the drug delivery system comprises:
a) an aminoglycoside, quinolone or macrolide antibacterial, and
b) an anti-Gram-negative lipopeptide antibacterial or other peptide antibacterial effective against Gram-negative bacteria.

Preferably, the lipopeptide antibacterial is colistin.

In a particular embodiment, the drug delivery system comprises the combination of a peptide antimicrobial (e.g. colistin) with an aminoglycoside antibacterial selected in the group consisting of spectinomycin, gentamicin, amikacin, arbekacin, kanamycin, neomycin, netilmicin, paromomycin, rhodostreptomycin, streptomycin, tobramycin and apramycin.

In a particular embodiment, the macrolide is azithromycin.

The invention further relates to a set of a first and a second compositions, wherein

the first composition comprises an aminoglycoside, macrolide or quinolone antibacterial, and

the second composition comprises a drug delivery system that is orally administered, and delivers specifically to the late ileum, caecum, or colon, and substantially avoids delivery to other areas of the gastrointestinal tract, wherein the drug delivery system comprises an anti-Gram-negative lipolipopeptide antibacterial or other peptide antibacterial effective against Gram-negative bacteria. In a particular embodiment, the first composition is a drug delivery system that is orally administered, and delivers specifically to the late ileum, caecum, or colon, and substantially avoids delivery to other areas of the gastrointestinal tract, wherein the drug delivery system comprises an aminoglycoside, macrolide or quinolone antibacterial.

In a particular embodiment, the antibacterials present in the composition or set according to the invention are selective for harmful (pathogenic) bacteria over helpful (beneficial) bacteria.

The invention also relates to the above composition or set of compositions for use in a method for providing elimination of Gram-negative resistant bacteria from the colon of a patient colonized by such bacteria. In particular, the method prevents the dissemination of the resistant bacteria in the environment (for instance, but not only upon admission at the hospital) and prevents the occurrence of an infection caused by these bacteria in the colonized patient (for instance but not only before surgical procedure).

The invention also relates to the above composition or set of compositions for use in a method for providing elimination of Gram-negative resistant bacteria from the colon of a patient at risk before he develops an actual infection.

The invention also relates to the above composition or set of composition, for use in a method of eliminating pathogenic microbes within the lumen of the intestinal tract, and minimizing the pathogenic alterations of the mucosa resulting from the action of compounds released by the infecting bacteria.

The invention further relates to the above composition or set of compositions, for use in a method for providing elimination of Gram-negative bacteria from the colon of farm animals, in particular wherein the colonic bacteria to be targeted are Shiga-toxin Escherichia coli (or STEC).

The above composition or set of compositions can also be used in a method for providing selective decontamination in a patient to control outbreaks of antibacterial-resistant Gram-negative infections, such as nosocomial infections, in hospitals. Said nosocomial infection may be caused by a) Gram-negative bacteria which are resistant to third generation cephalosporins by secretion of an extended spectrum beta-lactamase (ESBL) derived from the TEM or SHV beta-lactamase families, b) Gram-negative bacteria which are resistant to third generation cephalosporins by secretion of an extended spectrum beta-lactamase (ESBL) derived from CTX-M beta-lactamase family, or c) Gram-negative bacteria which are resistant to antibacterials by secretion of other types of enzymes such as carbapenemases of the KPC as well as other enzymatic families.

The invention further relates to the above composition or set of compositions, for use in a method of reducing the concentration of bacteria in the colon of a patient who has a colonic bacterial infection, or who is at risk of having a colonic bacterial infection. In this case, the composition according to the invention can further include a third active agent, for example an anti-inflammatory compound, an anti-histamine, an anti-cholinergic, an antiviral, an antimitotic, a diagnostic agent, or an immunosuppressive agent.

The invention also relates to a kit comprising:

a first composition comprising a drug delivery system that is orally administered, and

delivers specifically to the late ileum, caecum, or colon, and substantially avoids delivery to other areas of the gastrointestinal tract, wherein the drug delivery system comprises an anti-Gram-negative lipopeptide antibacterial or other peptide antibacterial effective against Gram-negative bacteria, and

a second composition comprising an aminoglycoside, macrolide or quinolone antibacterial.

The present invention will be better understood with reference to the following detailed description.

### DETAILED DESCRIPTION

The drug delivery compositions, and methods of treatment described herein, eliminate unwanted Gram-negative bacteria from the lower intestine and prevent emergence of resistance among these bacterial population.

In one embodiment, the compositions include
an anti-Gram-negative lipopeptide antibacterial, such as colistin or any other anti Gram-negative peptide antibacterial, and
a macro lide, quino lone, or aminoglycoside antibacterial, preferably a macrolide or aminoglycoside antibacterial.

The delivery composition will prevent any significant systemic absorption of the antibacterials thus avoiding or limiting any possibility of systemic toxic effect.

The compositions include a combination of antibacterial agents widely used for a number of years for the treatment of humans and animals, and therefore are well-known in terms of activity against Gram-negative bacteria as well as in terms of pharmacology and possible toxicity and side effects.

In addition, the combination of these two drugs prevents the emergence of resistant bacteria by selection of mutants resistant to any one of either of the components. Indeed, the association of drugs is an effective means of preventing the emergence of such mutants.

Each of the drugs, by themselves, has been shown to be able to eliminate from the intestinal tract Gram-negative bacteria which are susceptible to them.

Each of the drugs, by themselves, has been shown to select little resistant bacteria when they are used to eliminate Gram-negative bacteria from the intestinal tract.

In one embodiment, the macrolide, quino lone, or aminoglycoside antibacterial is administered in one formulation, and the peptide antibacterial is administered separately as colonic delivery formulations. In another embodiment, the combined antibacterials can be combined in the same formulation at a given efficient dose. The following description includes the best presently contemplated mode of carrying out the invention. This description is made for the purpose of illustrating the general principles of the inventions and should not be taken in a limiting sense.

The drug delivery systems described herein will be better understood with reference to the following detailed description, and the following definitions:

The terms “a”, “an” and “the” as used herein are defined to mean “one or more” and include the plural unless the context is inappropriate.

Unless defined otherwise, all technical and scientific terms used herein have the same meaning as commonly understood by one having ordinary skill in the art. Although other materials and methods similar or equivalent to those described herein can be used in the practice or testing of the present invention, as would be apparent to practitioners in the art, the preferred methods and materials are now described.

The terms “inhibit”, “inhibition”, “inhibitory”, and “inhibitor” all refer to a function of reducing a biological activity or function. Such reduction in activity or function can, for example, be in connection with a cellular component, e.g., an enzyme, or in connection with a cellular process, e.g., synthesis of a particular protein, or in connection with an overall process of a cell, e.g., cell growth. In reference to bacterial cell growth, for example, an inhibitory effect (i.e., a bacteria-inhibiting effect) may be bacteriocidal (killing of bacterial cells) or bacteriostatic (i.e., stopping or at least slowing bacterial cell growth). The latter slows or prevents cell growth such that fewer cells of the strain are produced relative to uninhibited cells over a given period of time. From a molecular standpoint, such inhibition may equate with a reduction in the level of, or elimination of, the transcription and/or translation of a specific bacterial target(s), or reduction or elimination of activity of a particular target biomolecule.

As used herein, “polymyxin” is defined as anti Gram-negative lipopeptides which are basic decapeptides containing a heptapeptide ring and a fatty acid chain in the N-terminal position. In addition to the lipopeptides, analogs thereof which no longer include the lipid moiety, but which retain the anti-Gram-negative efficacy, can be used.

The terms “lipopeptide” and “lipopolypeptide” are herein used interchangeably.

A “target” refers to a biomolecule in a bacteria that can be acted on by an active agent as described herein, thereby modulating, preferably inhibiting, growth or viability of a bacterial cell. In most cases such a target will be a nucleic acid sequence or molecule, or a lipopeptide or protein. However, other types of biomolecules can also be targets, e.g., membrane lipids and cell wall structural components.

1. Macrolide Antibacterials

The macrolides are a group of drugs (typically antibacterials) whose activity stems from the presence of a macrolide ring. A macrolide ring is a large macrocyclic lactone ring, to which one or more deoxy sugars, usually cladinose and desosamine, may be attached. The lactone rings are usually 14, 15 or 16-membered. Macrolides belong to the polyketide class of natural products.

There are several commonly-used macrolide antibacterials, including Azithromycin (Zithromax, Zitromax, Sumamed), Clarithromycin (Biaxin), Dirithromycin (Dynabac), Erythromycin, Roxithromycin (Rulid, Surlid, Roxid), Josamycin, Tylosin/tylocine (Tylan) and Tulathromycin

There are also several macrolide antibacterials in development, including Carbomycin A, Kitasamycin, Midecamicine/midecamicine acetate, Oleandomycin, Spiramycin, and Troleandomycin. Additional macrolides include roxithromycin, rokitamycin, miocamycin, flurithromycin, rosaramicin, and compounds designated ABT-229 or ABT-269.

2. Ketolides

Ketolides are a new class of antibacterials that are structurally related to the macrolides. Representative ketolides include Telithromycin (Ketek), Cethromycin, spiramycin, ansamycin, oleandomycin, carbomycin and tylocine.

Additional macrolides include those described in “Antibacterial Agents,” Chapter 30, André Bryskier, ed., ASM press, Washington (2005).

The macrolide antibacterials have been used in the past mainly for their systemic anti-gram positive activity and to treat infections caused by gram positive pathogenic bacteria such as staphylococci or streptococci instance. Indeed, the serum and tissue levels achieved with the first generation macrolides such as erythromycin are high enough to kill these gram positive organisms. However, they are not sufficient to kill Gram-negative organisms. Thus, these early macro lides were not used to treat systemic Gram-negative infections.

However, the situation is different in the lumen of the colon. There, the concentration of macrolides achieved is much higher than in the blood and tissues (for instance, in the order of magnitude of 1 mg/mL versus 1-5 μg/mL in the serum). The consequence is that the normal enterobacteria present in the colon are eliminated from the lumen during treatment with erythromycin. Thus, the molecule can be used for eliminating enterobacteria from the colon (Reduction of the aerobic Gram-negative bacterial flora of the gastro-intestinal tract and prevention of traveller's diarrhea using oral erythromycin. Andremont A, Tancrede C. Ann Microbiol (Paris). 1981 November-December; 132 B(3):419-27).

The selection of enterobacteria resistant to the high concentration of erythromycin present in the colon is an infrequent event. Thus, the drug is a good candidate to eliminate enterobacteria from the colon over a rather prolonged period of time, with a low likelihood of selecting resistant clones.

At the same time, the drug has activity on the other bacterial populations from the colonic ecosystem. However, the susceptible clones of these other bacterial populations are typically replaced very rapidly by resistant ones, resulting in the fact that an analysis of the composition of the colonic flora during erythromycin treatments, would show no major changes in terms of species present except for the elimination of enterobacteria.

In addition, it was observed that the flora that persisted in the colon during erythromycin treatment, although it is made of bacteria resistant to erythromycin, was keeping its property to prevent colonization by exogenous organisms, the so-called “resistance to colonization” (Effect of erythromycin on microbial antagonisms: a study in gnotobiotic mice associated with a human fecal flora. Andremont A, Raibaud P, Tancrede C. J Infect Dis. 1983 September; 148(3):579-87.). Thus, the drug is a good candidate to achieve elimination of Gram-negative bacteria from the colon. Indeed it was tested for such use in various clinical settings (Reduction of the aerobic Gram-negative bacterial flora of the gastro-intestinal tract and prevention of traveller's diarrhea using oral erythromycin. Andremont A, Tancrede C. Ann Microbiol (Paris). 1981 November-December; 132 B(3):419-27, Selective digestive decontamination by erythromycin-base in a polyvalent intensive care unit. de Champs C L, Guelon D P, Garnier R M, Poupart M C, Mansoor O Y, Dissait F L, Sirot J L. Intensive Care Med. 1993; 19(4):191-6.). However, it did not gain general acceptance for several reasons: erythromycin is a prokinetic agent acting on the stomach and the ileum, responsible for an accelerated transit time which was not acceptable in the patients that were to be decontaminated.

In addition, no measures were taken, in this use, to prevent the absorption of erythromycin in the upper part of the intestine, thus the possible systemic toxicity of erythromycin was still present. Lastly, erythromycin was used in relatively high doses, up to 3 g per day, doses which are difficult to absorb in severely ill patients such as those hopitalized in an intensive care unit (ICU).

Among the advantages of azythromycin over erythromycin are that azithromycin has no or very little prokinetic effect, so there will be no need to bother about that side effect, and second, its activity against Gram-negative bacteria that are to be eliminated from the colon is much stronger than that of erythromycin. A daily dose of 500 mg of Azithromycin is effective to eliminate Gram-negative enterobacteria from the colon, and lower doses are also effective, due to the high activity of Azithromycin. In addition, by targeting the lower part of the intestine the systemic absorption and systemic toxicity of Azithromycin will be avoided.

Mechanism of Action

The mechanism of action of the macrolides involves inhibition of bacterial protein biosynthesis by binding reversibly to the 50S subunit of the bacterial ribosome, thereby inhibiting translocation of the peptidyl tRNA. This action is mainly bacteriostatic, but can also be bactericidal at high concentrations. Macro lides tend to accumulate within leukocytes, and are therefore efficiently transported to the site of infection.

Resistance

The primary means of bacterial resistance to macrolides occurs by post-transcriptional methylation of the 23S bacterial ribosomal RNA. This acquired resistance can be either plasmid-mediated or chromosomal, i.e. through mutation, and results in cross-resistance to macrolides, lincosamides, and streptogramins (an MLS-resistant phenotype).

Two other types of acquired resistance rarely seen include the production of drug-inactivating enzymes (esterases or kinases) as well as the production of active ATP-dependent efflux proteins that transport the drug outside of the cell.

Side Effects

Macrolides exhibit enterohepatic recycling; that is, the drug is absorbed in the gut and sent to the liver, only to be excreted into the duodenum in bile from the liver. This can lead to a buildup of the product in the body, causing nausea. However, these side effects will be minimized by the local delivery to the colon, caecum, or ileum.

Azithromycin is a preferred macrolide, and can be used in any of its forms. As used herein, “azithromycin” means all amorphous and crystalline forms of azithromycin including all polymorphs, isomorphs, pseudomorphs, clathrates, salts, solvates and hydrates of azithromycin, as well as anhydrous azithromycin. Reference to azithromycin in terms of therapeutic amounts or in release rates in the claims is to active azithromycin, i.e., the non-salt, non-hydrated azalide molecule having a molecular weight of 749 g/mole.

Preferably, the azithromycin of the present invention is azithromycin dihydrate, which is disclosed in U.S. Pat. No. 6,268,489.

In alternate embodiments of the present invention, the azithromycin comprises a non-dihydrate azithromycin, a mixture of non-dihydrate azithromycins, or a mixture of azithromycin dihydrate and non-dihydrate azithromycins. Examples of suitable non-dihydrate azithromycins include, but are not limited to, alternate crystalline forms B, D, E, F, G, H, J, M, N, O, P, Q and R.

Azithromycin also occurs as Family I and Family II isomorphs, which are hydrates and/or solvates of azithromycin. The solvent molecules in the cavities have a tendency to exchange between solvent and water under specific conditions.

Therefore, the solvent/water content of the isomorphs may vary to a certain extent.

Azithromycin form B, a hygroscopic hydrate of azithromycin, is disclosed in U.S. Pat. No. 4,474,768.

Azithromycin forms D, E, F, G, H, J, M, N, O, P, Q and R are disclosed in U.S. Pat. No. 6,977,243.

Forms B, F, G, H, J, M, N, O, and P belong to Family I azithromycin and have a monoclinic P21 space group with cell dimensions of a=16. 30. 3 A, b=16. 2+0. 3 A, c=18. 40. 3 A and beta=1092°.

Form F azithromycin is an azithromycin ethanol solvate of the formula C₃₈H₇₂N₂₀ 2 H_{2O} 5 C₂H₅0H in the single crystal structure and is an azithromycin monohydrate hemi-ethanol solvate. Form F is further characterized as containing 2-5 wt % water and 1-4 wt % ethanol by weight in powder samples.

The single crystal of form F is crystallized in a monoclinic space group, P21, with the asymmetric unit containing two azithromycin molecules, two water molecules, and one ethanol molecule, as a monohydrate/hemi-ethanolate. It is isomorphic to all Family I azithromycin crystalline forms. The theoretical water and ethanol contents are 2.3 and 2.9 wt %, respectively.

Form G azithromycin has the formula C₃₈H₇₂N₂O₁₂. 5H₂O in the single crystal structure and is an azithromycin sesquihydrate. Form G is further characterized as containing 2.5-6 wt % water and <1 wt % organic solvent (s) by weight in powder samples. The single crystal structure of form G consists of two azithromycin molecules and three water molecules per asymmetric unit, corresponding to a sesquihydrate with a theoretical water content of 3.5 wt %. The water content of powder samples of form G ranges from about 2.5 to about 6 wt %.

The total residual organic solvent is less than 1 wt % of the corresponding solvent used for crystallization.

Form H azithromycin has the formula C₃₈H₇₂N₂O₁₂ H₂O 0.5 C₃H₈O₂ and may be characterized as an azithromycin monohydrate hemi-1,2 propanediol solvate. Form H is a monohydrate/hemi-propylene glycol solvate of azithromycin free base.

Form J azithromycin has the formula C₃₈H₇₂N₂O₁₂ H₂O 0.5 C₃H₇OH in the single crystal structure, and is an azithromycin monohydrate hemi-n-propanol solvate. Form J is further characterized as containing 2-5 wt % water and 1-5 wt % n-propanol by weight in powder samples. The calculated solvent content is about 3.8 wt % n-propanol and about 2.3 wt % water.

Form M azithromycin has the formula C₃₈H₇₂N₂O₁₂ H₂O 0.5 C₃H₇OH, and is an azithromycin monohydrate hemi-isopropanol solvate. Form M is further characterized as containing 2-5 wt % water and 1-4 wt % 2-propanol by weight in powder samples. The single crystal structure of form M would be a monohydrate/hemi-isopropranolate.

Form N azithromycin is a mixture of isomorphs of Family I. The mixture may contain variable percentages of isomorphs F, G, H, J, M and others, and variable amounts of water and organic solvents, such as ethanol, isopropanol, n-propanol, propylene glycol, acetone, acetonitrile, butanol, pentanol, etc. The weight percent of water can range from 1-5.3 wt % and the total weight percent of organic solvents can be 2-5 wt % with each solvent making up 0.5-4 wt %.

Form 0 azithromycin has the formula C₃₈H₇₂N₂O₁₂ 0.5 H₂O, 0.5 C₄H₉OH, and is a hemihydrate hemi-n-butanol solvate of azithromycin free base by single crystal structural data.

Form P azithromycin has the formula C₃₈H₇₂N₂O₁₂ H₂O 0.5 C₅H₁₂O and is an azithromycin monohydrate hemi-n-pentanol solvate.

Form Q is distinct from Families and)), has the formula C₃₈H₇₂N₂O₁₂ H₂O 0.5 C₄H₈O and is an azithromycin monohydrate hemi-tetrahydrofuran (THF) solvate. It contains about 4% water and about 4.5 wt % THF.

Forms D, E and R belong to Family in azithromycin and contain an orthorhombic P21 2121 space group with cell dimensions of a=8. 90. 4 A, b=12. 30. 5 A and c=45. 80. 5 A.

Form D azithromycin has the formula C₃₈H₇₂N₂O₁₂ H₂O C₆H₁₂ in its single crystal structure, and is an azithromycin monohydrate monocyclohexane solvate. Form D is further characterized as containing 2-6 wt % water and 3-12 wt % cyclohexane by weight in powder samples. From single crystal data, the calculated water and cyclohexane content of form D is 2.1 and 9.9 wt %, respectively.

Form E azithromycin has the formula C₃₈H₇₂N₂O₁₂ H₂O C₄H₈O and is an azithromycin monohydrate mono-THF solvate by single crystal analysis.

Form R azithromycin has the formula C₃₈H₇₂N₂O₁₂ H₂O C₅H₁₂0 and is an azithromycin monohydrate mono-methyl tert-butyl ether solvate. Form R has a theoretical water content of 2.1 wt % and a theoretical methyl tert-butyl ether content of 10.3 wt %.

Other examples of non-dihydrate azithromycin include, but are not limited to, an ethanol solvate of azithromycin or an isopropanol solvate of azithromycin. Examples of such ethanol and isopropanol solvates of azithromycin are disclosed in U.S. Pat. Nos. 6,365,574 and 6,245,903 and U.S. Pat. No. 6,977,243.

Additional examples of non-dihydrate azithromycin include, but are not limited to, azithromycin monohydrate as disclosed in U.S. Pat. No. 6,586,576, as well as International Application Publication Nos. WO 01/00640, WO 01/49697, WO 02/10181 and WO 02/42315.

Further examples of non-dihydrate azithromycin include, but are not limited to, anhydrous azithromycin as disclosed in U.S. Pat. Nos. 7,414,114, and 6,528,492.

Examples of suitable azithromycin salts include, but are not limited to, the azithromycin salts as disclosed in U.S. Pat. No. 4,474,768.

Preferably, at least 70 wt % of the azithromycin in the multiparticulates is crystalline. The degree of azithromycin crystallinity in the multiparticulates can be “substantially crystalline,” meaning that the amount of crystalline azithromycin in the multiparticulates is at least about 80%, “almost completely crystalline,” meaning that the amount of crystalline azithromycin is at least about 90%, or “essentially crystalline,” meaning that the amount of crystalline azithromycin in the multiparticulates is at least 95%.

The crystallinity of azithromycin in the multiparticulates may be determined using Powder X Ray Diffraction (PXRD) analysis. In an exemplary procedure, PXRD analysis may be performed on a Bruker AXS D8 Advance diffractometer. In this analysis, samples of about 500 mg are packed in Lucite sample cups and the sample surface smoothed using a glass microscope slide to provide a consistently smooth sample surface that is level with the top of the sample cup. Samples are spun in the (p plane at a rate of 30 rpm to minimize crystal orientation effects. The X-ray source (S/B KCua, A=1. 54 A) is operated at a voltage of 45 kV and a current of 40 mA. Data for each sample are collected over a period of from about 20 to about 60 minutes in continuous detector scan mode at a scan speed of about 12 seconds/step and a step size of 0.02°/step. Diffractograms are collected over the 20 range of 10° to 16°.

The crystallinity of the test sample is determined by comparison with calibration standards as follows. The calibration standards consist of physical mixtures of 20 wt %/80 wt % azithromycin/carrier, and 80 wt %/20 wt % azithromycin/carrier. Each physical mixture is blended together 15 minutes on a Turbula mixer. Using the instrument software, the area under the diffractogram curve is integrated over the 26 range of 10° to 16° using a linear baseline. This integration range includes as many azithromycin-specific peaks as possible while excluding carrier-related peaks. In addition, the large azithromycin-specific peak at approximately 10° 29 is omitted due to the large scan-to-scan variability in its integrated area. A linear calibration curve of percent crystalline azithromycin versus the area under the diffractogram curve is generated from the calibration standards.

The crystallinity of the test sample is then determined using these calibration results and the area under the curve for the test sample. Results are reported as a mean percent azithromycin crystallinity (by crystal mass).

Other macrolide antibacterials with similar properties to azithromycin can also be used. The desirable properties include long term-use in humans, once daily administration, low daily dose of administration (500 mg or less in adults), reduced intestinal side effects in comparison with the parental drug erythromycin, high efficacy against Gram-negative bacteria in vitro, proven efficacy to eliminate Gram-negative bacteria from the intestinal tract, low rate of selection of resistant Gram-negative bacteria in the intestinal tract even when used alone.

3. Aminoglycoside Antibacterials

An aminoglycoside is a molecule composed of a sugar group and an amino group. Several aminoglycosides are effective against Gram-negative bacteria, including amikacin, arbekacin, gentamicin, kanamycin, neomycin, netilmicin, paromomycin, rhodostreptomycin, streptomycin, tobramycin, spectinomycin and apramycin. Particularly preferred aminoglycosides include streptomycin, tobramycin, spectinomycin and gentamicin.

Aminoglycosides work by binding to the bacterial 30S ribosomal subunit (some work by binding to the 50S subunit), inhibiting the translocation of the peptidyl-tRNA from the A-site to the P-site and also causing misreading of mRNA, leaving the bacterium unable to synthesize proteins vital to its growth. Thus, it is believed that aminoglycosides kill bacteria by inhibiting protein synthesis as they bind to the 16S rRNA and by disrupting the integrity of the bacterial cell membrane.

It is also believed that the initial site of action is the outer bacterial membrane, where the cationic antibiotic molecules create fissures in the outer cell membrane, resulting in leakage of intracellular contents and enhanced antibiotic uptake. Aminoglycosides are useful primarily in infections involving aerobic, Gram-negative bacteria, such as Pseudomonas, Acinetobacter, and Enterobacter. Aminoglycosides are mostly ineffective against anaerobic bacteria (including Gram positive bacteria).

The toxicity of these agents is dose-related, and therefore every individual can be subject to these side effects (toxicity) provided the dose is sufficiently high. Because of their potential for ototoxicity and nephrotoxicity (kidney toxicity), aminoglycosides are administered in doses based on body weight. Vestibular damage, hearing loss and tinnitus are irreversible, so care must be taken not to achieve a sufficiently high dose. Concomitant administration of a cephalosporin may lead to increased risk of nephrotoxicity while administration with a loop diuretic increases the risk of ototoxicity. Blood drug levels and creatinine are monitored during the course of therapy, as individuals vary widely in the relationship between dose and plasma level. Serum creatinine measurements are used to estimate how well the kidneys are functioning and as a marker for kidney damage caused by these drugs. They may react with and prolong the actions of neuromuscular agents. Impaired renal function necessitates a reduced dose. Dosing and monitoring of aminoglycosides are routinely performed by hospital clinical pharmacists.

Aminoglycosides are not typically absorbed from the gut, so that in the present invention, when they are orally administered, and released directly in or near the colon, systemic toxicity can be largely avoided.

4. Quinolone Antibacterials

The quinolones are a family of synthetic broad-spectrum antibiotics. The parent of the group is nalidixic acid. The majority of quinolones in clinical use belong to the subset of fluoroquinolones, which have a fluorine atom attached the central ring system, typically at the 6-position.

Quinolones have a broad spectrum of antimicrobial activity, as well as a unique mechanism of action resulting in inhibition of bacterial DNA gyrase and topoisomerase IV. Quinolones typically include a quinoline ring, and fluoroquinolones (also within the scope of the invention) include a fluorine atom at C6.

Mechanism

Quinolones and fluoroquinolones are chemotherapeutic bactericidal drugs, eradicating bacteria by interfering with DNA replication. Quinolones inhibit the bacterial DNA gyrase or the topoisomerase IV enzyme, thereby inhibiting DNA replication and transcription. DNA gyrase is the target for many Gram-negative bacteria, making quinolones effective against these bacteria.

Bacterial Resistance

Resistance to quinolones can evolve rapidly, even during a course of treatment. Numerous pathogens, including Staphylococcus aureus, enterococci, and Streptococcus pyogenes now exhibit resistance worldwide.

Quinolones that can be used include, without limitation, cinoxacin (Cinobac), flumequine (Flubactin), nalidixic acid (NegGam, Wintomylon), oxolinic acid (Uroxin), piromidic acid (Panacid), pipemidic acid (Dolcol), rosoxacin (Eradacil), ciprofloxacin (Ciprobay, Cipro, Ciproxin), enoxacin (Enroxil, Penetrex), fleroxacin (Megalone, Roquinol), lomefloxacin (Maxaquin), nadifloxacin (Acuatim, Nadoxin, Nadixa), norfloxacin (Lexinor, Noroxin, Quinabic, Janacin), ofloxacin (Floxin, Oxaldin, Tarivid), pefloxacin (Peflacine), rufloxacin (Uroflox), balofloxacin (Baloxin), gatifloxacin (Tequin) (Zymar), grepafloxacin (Raxar), levofloxacin (Cravit, Levaquin), moxifloxacin (Avelox, Vigamox), pazufloxacin (Pasil, Pazucross), sparfloxacin (Zagam), temafloxacin (Omniflox), tosufloxacin (Ozex, Tosacin), clinafloxacin, gemifloxacin (Factive), sitafloxacin (Gracevit), trovafloxacin (Trovan), prulifloxacin (Quisnon), garenoxacin (Geninax), ecinofloxacin, marbofloxacin and delafloxacin.

5. Peptide Antibacterials

As used herein, peptide antibacterials include those with specific activity against Gram-negative bacteria. Primarily, the peptide antibacterials are anti-Gram-negative lipopeptide antibacterials. These include colistin and other molecules made of a cyclic ring of peptide and of fatty tail. Many of these are of bacterial origin, although there are some synthetic examples as well. The lipid (fatty) tail may or may not be present, so long as the molecule has significant anti-Gram-negative activity.

Colistin, for example, is isolated from the fermentation of Bacillus polymixa. Additional examples include those described in Antibacterial agents edited by André Bryskier Published by ASM press, Washington, in 2005, Chap 30.

Colistin can be preferred for a number of reasons. However, real efficacy has never been clearly shown for intestinal “disinfection” following oral administration, probably for two main reasons. First, to treat such a patient, one may also need antibacterial activity in the mucosa because of a possible ongoing invasive process with some bacterial species as colistin is not absorbed at all, it is unlikely that any tissue concentration of the antibiotic would build up. Second, the doses used were probably too low to achieve adequate intraluminal concentrations in the colon.

Over time, the oral use of colistin has faded. However, in the present application, with specific delivery to or near the colon, colistin's strong anti-Gram-negative bacteria activity and lack of systemic absorption after oral administration are major advantages.

While not wishing to be bound to a particular theory, it is believed that part of the colistin is degraded in the jejunum, and thus the efficacious concentration in the colon can only be achieved with high doses. By protecting colistin in the upper part of the intestine via proper formulation, one can both prevent its possible local toxicity there, and also prevent its partial degradation. This would allow one to administer lower doses than the 600 mg daily dose currently necessary to achieve decontamination in adults.

Anti-Gram-negative peptide antibacterials are well-known (See, for example, Hancocket al. Adv. Microb. Physiol. 37:135-175; Kleinkauf et al., 1988. Crit. Rev. Biotechnol. 8:1-32; and Perlman and Bodansky. 1971. Ann. Rev. Biochem. 40:449-464), and fall into two classes, non-ribosomally synthesized peptides, such as gramicidins, polymyxins, bacitracins, glycopeptides, etc., and ribosomallysynthesized (natural) peptides. Representative antibacterials that are used commercially include colistin (also known as colistin or polymyxin E), bacitracin, gramicidin S, and polymyxin B.

The best peptides have Minimum Inhibitory Concentrations (MICs) of 1 to 8 μg/ml against a wide range of Gram-negative bacteria, including some of the most difficult to treat, antibacterial-resistant pathogens. They are bactericidal with very rapid killing kinetics, even around the MIC, and there are very few naturally-resistant bacteria.

The preferred lipopeptide antibacterial agent is colistin, but the antibacterial agent can be any other lipopeptide antibacterial agent with similar properties. Such properties include possible long term-use in humans, low daily dose of administration (600 mg or less in adults), reduced intestinal side effects, high efficacy against Gram-negative bacteria in vitro, proven efficacy to eliminate Gram-negative bacteria from the intestinal tract, low frequency of selection of resistant Gram-negative bacteria in the intestinal tract even when used alone.

Colistin (polymyxin E) is a polymyxin antibiotic produced by certain strains of Bacillus polymyxa. colistinus. Colistin is a mixture of cyclic polypeptides colistin A and B. Colistin is effective against most Gram-negative bacilli and is used as a polypeptide antibiotic.

There are two forms of colistin available commercially: colistin sulfate and colistimethate sodium (colistin methanesulfonate sodium, colistin sulfomethate sodium).

Colistin has systemic toxicity which may in part be due to the lipid tail appended to the nonapeptide, but even the deacylated derivative of polymyxin (polymyxin B nonapeptide) tends to be too toxic for human systemic use. (Wolinsky E, Hines J D. (1962). “Neurotoxic and nephrotoxic effects of colistin in patients with renal disease”. N Engl J Med 266: 759-68; Koch-Weser J, Sidel V W, Federman E B, et al. (1970). “Adverse effects of sodium colistimethate. Manifestations and specific reaction rates during 317 courses of therapy”. Ann Intern Med 72: 857-68; Li J, Nation R L, Milne R W, et al. (2005). “Evaluation of colistin as an agent against multi-resistant Gram-negative bacteria”. Int J Antimicrob Agents 25: 11-25.)

Indeed, the nonacylated cyclic decapeptide gramicidin S is also quite toxic, causing erythrocyte lysis at concentrations only threefold higher than the MIC for many bacteria. For this reason, these peptides have typically been restricted to topical applications. The present invention proposes to take advantage of their strong antibacterial activity, without resulting in systemic toxicity, by delivering them locally to the colon.

The polymyxin complexes are part of the lipopeptides, members of the subclass of the cyclic lipopeptides and described as lipopeptides active against Gram-negative bacteria (see, for example, Antibacterial Agents, Chapter 30, André Bryskier, ed., ASM press, Washington (2005).

The anti-Gram-negative lipopeptides (also referred to herein as members of the Polymyxin complex) include eight different members: Circulins A and B, Polymyxin A (M), Polymyxin B1 and B2, Polymyxin (or P), Polymyxin D1/D2, Polymyxin E1 (colistinA), Polymyxin S1 and Polymyxin T1.

Of these, polymyxin E1 (colistin A) is preferred.

Antibacterial Targeted-Delivery

By providing delivery of these antibacterial drugs to the lower part of the intestinal tract, in particular to the distal ileum, caecum or colon, the compositions described herein can provide the antibacterial drugs at efficient concentrations to treat Gram-negative bacteria while limiting adverse effects in the upper part of the gastro-intestinal tract (in particular gastric side effects), and limiting their possible systemic toxicity.

Moreover, by delivering antibacterials directly to the distal ileum/caecum/colon, one can prevent or significantly limit their absorption into the bloodstream, and hence their systemic effect. This is particularly advantageous if one wishes to solely target microorganisms in the lumen of the gastrointestinal tract, because it enables (i) to maximize the antibacterial concentration at this location, and (ii) to avoid or limit any other effects of the antibacterial such as generating resistant bacteria at other sites and in particular at sites such as the respiratory tract where one may wish to use those antibacterials to treat infections.

The drug delivery systems are administered in an effective amount suitable to provide the adequate degree of treatment or prevention of the disorders for which the compounds are administered.

The efficient amounts of these compounds are typically below the threshold concentration required to elicit any appreciable side effects.

The compounds can be administered in a therapeutic window in which some the disorders are treated and certain side effects are avoided. Ideally, the effective dose of the compounds described herein is sufficient to provide the desired effects in the colon but is insufficient (i.e., is not at a high enough level) to provide undesirable side effects elsewhere in the body.

Most preferably, effective doses are at very low concentrations, where maximal effects are observed to occur, with minimal side effects, and this is optimized by targeted colonic delivery of the active agents. The foregoing effective doses typically represent that amount administered as a single dose, or as one or more doses administered over a 24-hour period.

The toxicity and therapeutic efficacy of the active agents described herein can be determined by standard pharmaceutical procedures in cell cultures or experimental animals, e.g., by determining the LD₅₀, (the dose lethal to 50% of the population) and the ED₅₀ (the dose therapeutically effective in 50% of the population). The dose ratio between toxic and therapeutic effect is the therapeutic index and can be expressed as the ratio between LD₅₀ and ED₅₀. Compounds which exhibit high therapeutic indices are preferred. The data obtained from these cell culture assays and animal studies can be used in formulating a dosage range that is not toxic for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1, p. 1).

Dosage amount and interval may be adjusted individually to provide levels of the active compounds in the colon which are sufficient to maintain therapeutic effect. Preferably, therapeutically effective levels will be achieved by administering multiple doses each day. One of skill in the art will be able to optimize therapeutically effective local dosages without undue experimentation.

Representative dosages (human daily doses) for the antibacterials used in the invention are around 0,2 to 200 mg/Kg/day, in particular around 4 to 50 mg/Kg/day. For example, colistin can be administered at a daily dose of around 2 to 12 mg/Kg/day, in particular around 10 mg/Kg/day. Representative dosages (human daily doses) for a macrolide antibacterial are around 2 to 10 mg/Kg/day, in particular around 7 mg/Kg/day. Representative dosages (human daily doses) for aminoglycosides are around 2 mg/Kg to 50 mg/Kg per day.

Representative dosages (human daily doses) for colistin are around 600 mg, and for a macrolide antibiotic are around 500 mg.

### Types of Colonic Delivery Systems

1. pH Responsive Delivery

There is a pH gradient in the gastrointestinal tract with values ranging from 1.2 in the stomach through 6.6 in the proximal small intestine to a peak of about 7.5 in the distal small intestine. This pH differential between the stomach and small intestine has historically been exploited to deliver drugs to the small intestine by way of pH sensitive enteric coatings. These polymer coatings are recalcitrant to the acidic conditions of the stomach but ionise and dissolve above a certain threshold pH found in the small intestine. Thus, it is also possible to apply this concept to deliver drugs to the terminal ileum or colon by use of enteric polymers with a relatively high threshold pH for dissolution. The most commonly used polymer for this purpose are copolymers of methacrylic acid, methyl methacrylate and methyl acrylate, which dissolves at a pH of greater than 7 (Eudragit™, Rohm Pharma, Darmstadt, Germany) but there are other polymers that can be used form different manufacturers.

Pharmaceutical products are available on the market which uses these technologies such 5-amino salicylic acid formulations for the treatment of ulcerative colitis (Asacol™, Ipacol™, Clayersal™)

2. Time Responsive Delivery

Time dependent dosage forms are formulated to release their drug load after a predetermined lag time. While not site-specific delivery systems per se, it has been suggested that colonic targeting can be achieved by incorporating a lag time into the formulation equivalent to the mouth to colon transit time. A nominal lag time of 5 hours is usually considered sufficient, since small intestinal transit has been considered relatively constant at 3 to 4 hours. A number of systems have been developed based on this principle, with one of the earliest being the Pulsincap® device. This device consists of a non-disintegrating half capsule shell sealed at the open end with a hydrogel plug. The plug hydrates on contact with gastrointestinal fluids and swells to an extent that it is expelled from the capsule body, thus releasing the drug. Usually the time it takes the hydrogel plug to hydrate and eject from the capsule shell defines the lag time prior to drug release and hence by altering the composition and size of the hydrogel plug, it is possible to achieve drug release after varying lag times.

3. Pressure Responsive Delivery

A pressure-controlled colon delivery capsule (PC DC) has recently been described (Hu et al. 1187-93). This novel delivery system uses the increase in pressure of the luminal contents of the colon resulting from the reabsorption of water in the region. The PCDC is composed of drug, dispersed in suppository base, coated with the hydrophobic polymer ethylcellulose. Once swallowed, the temperature of the body causes the suppository base to melt and increase in volume, and the system resembles a liquid-filled ethylcellulose balloon. The balloon is able to withstand the luminal pressure of the small intestine resulting from muscular contraction of the gut wall (peristalsis), but will rupture when subjected to the pressure of the more intense contractions of the colon and contents of thicker viscosity.

4. Bacteria Responsive Delivery

These systems use substrates that are specifically degraded by enzymes produced by bacteria present in the colonic region, but not by gastro-intestinal enzymes produced by the host. Many of these polymers are already used as excipients in drug formulations, or are constituents of the human diet and are therefore generally regarded as safe. Although specifically degraded in the colon, many of these polymers are hydrophilic in nature, and swell under exposure to upper gastrointestinal conditions, which would result in premature drug release. To overcome this problem the natural polysaccharides are either chemically modified or mixed with hydrophobic, water-insoluble polymers. This has the effect of limiting the swelling in the upper gastrointestinal tract, but still permitting a partial solubilisation of the matrix or coating in the colon due to bacterial degradation resulting in drug release. The number of polysaccharides investigated to date is large, and a include amylose, chitosan, chondroitin sulphate, dextran, guar gum, inulin and pectin.

Compositions for Delivery to the Colon, Ileum, and/or Caecum

The compositions described herein are intended to deliver the lipopeptide antibacterial agent and the macrolide, quinolone or aminoglycoside antibacterial agent via oral administration, but specific delivery to the colon, caecum or the distal ileum is intended.

To achieve successful colonic delivery, a drug needs to be protected from absorption and/or the environment of the upper gastrointestinal tract (GIT) and then be released into the distal ileum, caecum and proximal colon, which is considered the optimum site for colon-targeted delivery of drugs. Moreover, drug release for both antibacterials can be modulated (prolonged delivery) to deliver the drug all along the colon to achieve best decontamination efficacy.

While providing delivery to the late ileum and colon, the compositions of the invention provide (i) protection of the antibacterial in the upper part of the gastrointestinal tract (GIT) from degradation, (ii) prevention of its systematic absorption in the upper part of the GIT, hence minimizing systematic diffusion of the antibacterial in unwanted tissues (upper respiratory tract for example) and (iii) release into the distal ileum, caecum and proximal colon, which is herein considered the optimum site for delivery of antibacterials for decontamination purposes.

In one embodiment, release of antibacterial can be modulated to offer prolonged antibacterial release all along the colon to achieve best decontamination efficacy. In another embodiment, release of the antibacterial is immediate when reaching the late ileum or colon.

In the compositions described herein,
the macro lide, quinolone or aminoglycoside antibacterial and
the anti Gram-negative lipopeptide antibacterial (or other peptide antimicrobial effective against Gram-negative bacteria)

can be combined in a weight ratio of 1/1 or in any other ratio with similar efficacy. For example, a weight ratio of macro lide, quinolone or aminoglycoside antibacterial to lipopeptide/peptide antibacterial can range between about 1/99 and about 99/1.

The dosage of the pharmaceutical compositions comprising the compounds (or antibacterials) of the invention can vary depending upon the age and mass of the subject being treated, the particular compound being administered, and the like.

The determination of dosage ranges and optimal dosages for a particular subject is within the ability of one of ordinary skill in the art.

In general, these compounds are most desirably administered in doses ranging from about 0.2 mg per kg body weight per day (mg/kg/day) to about 200 mg/kg/day in single or divided doses (i.e., from 1 to 4 doses per day), although variations will necessarily occur depending upon the species, weight and condition of the subject being treated and the particular route of administration chosen. However, a dosage level that is in the range of about 4 mg/kg/day to about 50 mg/kg/day is most desirably employed.

Variations may nevertheless occur depending upon the species of mammal, fish or bird being treated and its individual response to said medicament, as well as on the type of pharmaceutical formulation chosen and the time period and interval at which such administration is carried out.

In some instances, dosage levels below the lower limit of the aforesaid range may be more than adequate, while in other cases still larger doses may be employed without causing any harmful side effects, provided that such larger doses are first divided into several small doses for administration throughout the day.

The active compounds may be administered alone or in combination with pharmaceutically acceptable carriers or diluents by the routes previously indicated, and such administration may be carried out in single or multiple doses. More particularly, the active compounds may be combined with various pharmaceutically acceptable inert carriers. Such carriers include solid diluents or fillers, sterile aqueous media and various non-toxic organic solvents, etc. Moreover, oral pharmaceutical compositions can be suitably sweetened and/or flavored. In general, the active compounds are present in such dosage forms at concentration levels ranging from about 5.0% to about 70% by weight.

In one embodiment, the compositions are prepared in such a manner that the active agents are encapsulated in inactive non toxic layers (for example polymer layers), which let them become fully active and dispersed in the lower part of the ileum, the caecum, and/or the colon, where they function to eliminate colonizing resistant bacteria. They can also prevent contact of the active agents with the mucosa of the gastro-intestinal tract in the stomach, duodenum, the jejunum and part of the ileum, thus preventing direct toxicity or side effect at these levels.

In this embodiment, significant absorption by the intestinal mucosa and systemic dissemination in any other part of the body will be prevented, minimizing or eliminating secondary effects observed with the non-embodied forms of the drugs.

Various aspects of these drug delivery compositions are described in more detail below. Among the various strategies for targeting orally administered drugs to the colon, one can cite for example, coating of solid dosage forms with pH-sensitive polymers, timed-release polymer systems, encapsulation within polymers which are specifically degraded by colonic bacteria and to a lesser extent bioadhesive systems, osmotic controlled drug delivery systems or covalent linkage of a drug with a carrier. The concept of pH-sensitive delivery systems is mainly based on the fact that pH conditions vary continuously along the gastrointestinal tract reaching a maximum in the late ileum.

Time-dependent drug delivery systems are based on polymers that will take a define time to dissolve, releasing the drug after 3-4 h after oral administration.

Polymers that can be degraded by enzymes produced by microorganisms of the colonic commensal flora, such as azo-crosslinked polymers can be used to target drugs to colon. Drugs can be released from azo-polymer coated dosage forms, after reduction and subsequent cleavage of the azo bonds by the azoreductase enzymes present in the colonic microflora. The presence of azo-reductase enzymes in the colon is important to the drug release from azo-crosslinked polymers as well as azo-bonded prodrugs.

Certain plant polysaccharides such as pectin, amylose, inulin, and guar gum remain unaffected in the presence of gastrointestinal enzymes, and can be used to formulate colon-targeted drug delivery systems. Natural polysaccharides, particularly pectin, can be used to deliver drugs specifically to the colon. These polysaccharides remain intact in the physiological environment of stomach and small intestine. However, once the dosage form enters into the colon, it is acted upon by polysaccharidases produced by microorganisms of the colonic commensal flora, such as pectinases, which degrade the polysaccharides and release the drug into the colon.

It may be important to protect the polysaccharides before they enter the stomach and small intestine, because the hydrophilic properties of certain polysaccharides cause them to swell. One way to protect the polysaccharides is either to crosslink the polysaccharides, for example, by ionic crosslinking with a divalent ion such as a zinc or calcium ion, or to coat them with a cationic polymer. In order to encapsulate actives, certain polysaccharide polymers such as pectin must be crosslinked via divalent cations such as calcium and zinc.

Another way is to use a protective coat (also referred to as an enteric coating), for example, using a pH-appropriate polymer, such as certain Eudragit polymers known for this purpose.

Formulations coated with enteric polymers release the active agents when the pH progresses towards the alkaline range. Multicoated formulations pass through the stomach, and release the active agents after a lag time of around three to five hours, equivalent to small intestinal transit time.

Redox-sensitive polymers and bioadhesive systems have also been used to deliver the drugs into the colon.

Glycosidase activity of the colonic microflora can be used to liberate drugs from glycosidic prodrugs.

Drug delivery vehicles coated with a bioadhesive polymer that selectively provides adhesion to the colonic mucosa may release drug in the colon.

Various aspects of these drug delivery vehicles are described in more detail below.

In one embodiment, the macrolide, quinolone or aminoglycoside antibacterial and peptide antibacterial can be combined in the same targeted drug delivery system. In another embodiment, the macrolide, quinolone or amino glycoside antibacterial is formulated in a first targeted drug delivery system and the peptide antibacterial is formulated in a second, different from the first, targeted drug delivery system. In a further embodiment, the peptide antimicrobial is formulated in a targeted drug delivery system and the macrolide, quinolone or aminoglycoside antibacterial is not formulated in a targeted drug delivery system.

Pectin

In one embodiment, the drug delivery systems are designed to be orally administered, but deliver the active agents specifically to the colon, and substantially nowhere else in the gastrointestinal tract. Pectin pellets are one example of a formulation that can deliver active agents specifically to the colon.

The pectin pellets are formed from pectin, crosslinked by calcium, zinc or other metal ions, and can be coated with a cationic polymer and/or further coated with Eudragit® polymers. The pectin pellets may also encapsulate one or more active agents.

The stability and protection of pectin in gastric medium and intestinal medium can be ensured by coating with gastro-resistant polymers such as Eudragit® polymer or other polymer coatings. In contrast, uncoated pellets of pectin are typically not stable in such an environment and may not adequately protect their contents against degradation and/or inactivation. The polymer coatings ensure that the pectin pellets resist long enough so that their contents are able to reach the colon intact.

Pectin is a polysaccharide isolated from the cellular walls of superior plants, used widely in the agricultural food industry (as a coagulant or thickener for jams, ice creams and the like) and pharmaceutics. It is polymolecular and polydisperse. Its drug delivery system varies depending on the source, extraction conditions and environmental factors.

Pectins are principally composed of linear chains of beta-1,4-(D)-galacturonic acid, at times interspersed by units of rhamnose. The carboxylic groups of galacturonic acid can be partially esterified to yield methylated pectins. Two types of pectins are distinguished according to their degree of methylation (DM: number of methoxy groups per 100 units of galacturonic acid):
highly methylated pectin (HM: high methoxy) where the degree of methylation varies between 50 and 80%. It is slightly soluble in water and forms gels in acidic medium (pH<3.6) or in the presence of sugars;
weakly methylated pectin (LM: low methoxy), with a degree of methylation varying from 25 to 50%. More soluble in water than HM pectin, it gives gels in the presence of divalent cations such as Ca²⁺ or Zn²⁺ ions. Indeed, Ca²⁺ ions form “bridges” between the free carboxylated groups of galacturonic acid moities. The network that is formed has been described by Grant et al. under the name of <<egg-box model>> (Grant G. T. et al. (1973) Biological interactions between polysaccharides and divalent cations: the egg-box model, FEBS Letters, 32, 195).

There are also amidated pectins. Treatment of pectin by ammonia transforms some methyl carboxylate groups (—COOCH₃) into carboxamide groups (—CONH₂). This amidation confers novel properties to the pectins, in particular better resistance to variations in pH. Amidated pectins tend to be more tolerant to the variations in pH, and have also been studied for the manufacture of matricial tablets for colonic delivery (Wakerly Z. et al. (1997) Studies on amidated pectins as potential carriers in colonic drug delivery, Journal of Pharmacy and Pharmacology. 49, 622).

Pectin is degraded by enzymes originating from higher plants and various microorganisms (fungi, bacteria, and the like) among which bacteria from the human colonic flora. The enzymes produced by the microflora encompass a mixture of polysaccharidases, glycosidases and esterases.

Metal Cations

Divalent cations from various salts can be used to crosslink pectin. Examples include zinc sulfate, zinc chloride, and zinc acetate. Other di-, tri-, or polyvalent ions can be used such as calcium salts.

Eudragit® Polymers

The coating of drug-loaded cores such as tablets, capsules, granules, pellets or crystals offers many advantages, such as higher physicochemical stability, better compliance and increased therapeutic efficacy of the active agents. Indeed, the effectiveness of a medication depends not only on the actives it contains, but also on formulation and processing.

Poly(meth)acrylates have proven particularly suitable as coating materials. These polymers are pharmacologically inactive, i.e., are excreted unchanged. EUDRAGIT® is the trade name for copolymers derived from esters of acrylic and methacrylic acid, whose properties are determined by functional groups. The individual EUDRAGIT® grades differ in their proportion of neutral, alkaline or acid groups and thus in terms of physicochemical properties. The skillful use and combination of different EUDRAGIT® polymers offers ideal solutions for controlled drug release in various pharmaceutical and technical applications. EUDRAGIT® provides functional films for sustained-release tablet and pellet coatings. The polymers are described in international pharmacopeias such as Ph. Eur., USP/NF, DMF and JPE. EUDRAGIT® polymers can provide the following possibilities for controlled drug release:
Gastrointestinal tract targeting (gastroresistance, release in the colon)
Protective coatings (taste and odor masking, protection against moisture)
Delayed drug release.

EUDRAGIT® polymers are available in a wide range of different concentrations and physical forms (aqueous solution, aqueous dispersion, organic solution, solid substances).

The pharmaceutical properties of EUDRAGIT® polymers are determined by the chemical properties of their functional groups. A distinction is made between
poly(meth)acrylates, soluble in digestive fluids (by salt formation) EUDRAGIT® L, S, FS and E polymers with acidic or alkaline groups enable pH-dependent release of the active agent.

Applications: from simple taste masking via resistance solely to gastric fluid, to controlled drug release in all sections of the intestine
poly(meth)acrylates, insoluble in digestive fluids EUDRAGIT® RL and RS polymers with alkaline and EUDRAGIT® NE polymers with neutral groups enable controlled time release of the active by pH-independent swelling.

Enteric EUDRAGIT® coatings provide protection against drug release in the stomach and enable controlled release in the intestine. Targeted drug release in the gastrointestinal tract is recommended for particular applications or therapeutic strategies, for example when the agent is sparingly soluble in the upper digestive tract, or when the agent may be degraded by gastric fluid. Secondly, this dosage form is very patient-friendly as it does not stress the stomach and the number of doses of the therapeutic agent can be considerably reduced, thanks to prolonged delivery. The dominant criterion for release is the pH-dependent dissolution of the coating, which takes place in a certain section of the intestine (pH 5 to over 7) rather than in the stomach (pH 1-5). For these applications, anionic EUDRAGIT® grades containing carboxyl groups, can be mixed with each other. This makes it possible to finely adjust the dissolution pH, and thus to define the drug release site in the intestine. EUDRAGIT® L and S grades are suitable for enteric coatings. EUDRAGIT® FS 30 D is specifically used for controlled release in the colon.

Application benefits of enteric EUDRAGIT® coatings include:
pH-dependent drug release
protection of actives sensitive to gastric fluid
protection of the gastric mucosa from aggressive actives
increase in drug effectiveness
good storage stability
controlled release in the colon/GI targeting
   The composition of the above referred to Eudragit® polymers is known to the skilled artisan and may be found, in particular, in US 2008/0206350 (U.S. Ser. No. 12/034,943).

Other Types of Colonic Delivery

In addition to pectin pellets, other drug delivery systems are known to provide delivery to the colon. For example, as mentioned above certain Eudragit® polymers are known to dissolve at the pH in the colon, and are used to formulate drug delivery systems for colonic delivery. Mucoadhesive polymers are also known to be used to deliver agents to the colon. The mucoadhesive polymers can be coated onto microparticles or nanoparticles, and when administered, and the particles reach the colon, they adhere to the mucous membrane. When adhered, the compositions can degrade over time and release the active agents. Representative bioadhesive polymers, and drug delivery systems, are described in U.S. Pat. No. 6,365,187 to Edith Mathiowitz.

Another way to control the timing of delivery is to use a polymer coating like shellac. See, for example, Sinha and Kumria, “Coating polymers for colon specific drug delivery: A comparative in vitro evaluation,” Acta Pharm,. 53 (2003) 41-47. Sinha and Kumria teach that at a coat concentration of 3%, shellac provided a polymer coating for colon-specific drug delivery. Shellac is an abundantly available natural polymer, and the study showed that it can provide site-specific drug delivery. Further, variation in shellac coat thickness can facilitate drug delivery to terminal ileum, distal or proximal colon.

Other Types of Gastro-Intestinal Delivery

In some embodiments, the delivery systems are intended to deliver the active agents to a portion of the gastrointestinal tract other than the colon, and past the stomach (where the active agents can be metabolized). For example, one can deliver agents to the distal jejunum, the proximal ileum, or directly to the ileum. Such a formulation minimizes release of the agent in the upper part of the small intestine, i.e. above the distal jejunum.

In these embodiments, the pectin pellets, which specifically administer agents to the colon, would ideally not be used. Other formulations for delivery to other locations in the gastrointestinal tract are known to those of skill in the art, and intended to be part of the invention described herein. For example, Krishnamachari et al. (Int. J. Pharm, 338(1-2):238-237 (2007)) discloses microparticles with non-enzymatically degrading poly(dl-lactide-co-glycolide) (PLGA) core that deliver agents in a site specific manner to both the distal ileum and colon.

In a particular embodiment, the invention relates to a composition comprising a mixture of
a peptide antibacterial effective against Gram-negative bacteria, preferably an anti Gram-negative lipopeptide such as a polymyxin, and most preferably colistin, and
an aminoglycoside, macrolide or quinolone antimicrobial, said mixture being in the form of a solid dosage form such as a tablet, granule or pellet (a compact mixture obtainable, for example, by an extrusion spheronization process or tabletting process) said composition being further coated for delivery to the late ileum, caecum or colon.

The invention further relates to a kit comprising at least two different compositions, wherein
a first composition comprises a peptide antibacterial effective against Gram-negative bacteria, preferably an anti Gram-negative lipopeptide such as a polymyxin, and most preferably colistin, said first composition being in the form of a solid dosage form such as a tablet, granule or pellet, said first composition being further coated for delivery of the antibacterial contained therein to the late ileum, caecum or colon, and
a second composition comprising an aminoglycoside, macrolide or quinolone antimicrobial, said second composition being in the form of a solid dosage form such as a tablet, granule or pellet, said second composition being further coated for delivery of the antibacterial contained therein to the late ileum, caecum or colon.

In some embodiments, the drug delivery system of the invention comprises a core and one or more coatings around the core, the coatings being provided for delayed delivery of the content of the drug delivery system in the late ileum, caecum or colon.

The core can be made of inert particles made of microcrystalline cellulose or sugar and is typically a commercially available microcrystalline cellulose pellet such as Cellets™ or Celpheres™ or Ethispheres™ and Suglets™ for sugar-based pellets for example. The size of the core unit typically has a diameter within the range of 60-2000 microns, preferably the diameter of the core unit is within the range of 700-1000 microns. Generally the core unit constitutes 50-90 wt % of the total weight of the particles composition. In some embodiments, the core unit is 70-90 wt %, while in other embodiments it is preferably 60-70 wt % of the total weight of the particles composition. One benefit of using a unit core is that coating thereof is relatively easy such that hardly any erosion or dissolving of the core unit occurs during coating. Due to this, the coating rate may be increased (e.g., in comparison with sugar cores or “non-pareils”) which reduces the process time and therewith the production costs without adversely affecting the quality of the particles. Also, the thickness of the coat layers on the particles may be easily controlled by the coating and drying time. The calculated yields of the process are more reliable since hardly any core material is lost during the different coating processes.

When the core is made of an inert particle, this core is further coated with a composition comprising the antibacterial or mixture of antibacterials. The composition comprising the antibacterial(s) is prepared from a water or organic solution of an adequate concentration of the antibacterial(s), sprayed onto the inert core typically using a binder such as a hydrophilic polymer. Convenient hydrophilic polymer binders include HPMC. Among the commercially available grades, a low viscosity HPMC is generally desired such as Methocel E5™. However, any hydrophilic polymer having a sufficient binding property such as PVP, starch, hydrophilic cellulose derivatives, hydrophilic acrylate or methacrylate polymers can be used. The drug layer typically constitutes 2-70 wt % of the total weight of the particles composition. The active agents generally constitute 10-70 wt % of the total weight of the particles.

In this embodiment, the drug delivery system comprises a core coated with a layer comprising the active agents (the antibacterial or mixture of antibacterials). An external layer is provided around the active agent layer, said external layer providing delayed release of the active agents (the antimicrobials) in the late ileum, the caecum or the colon. Any of the delayed delivery systems described above can be used. For example, the external layer can provide pH-dependent, time responsive, pressure responsive or bacteria responsive delivery.

In a particular embodiment of the invention, an inert core as described above is coated with a layer of antibacterial agent(s) (for example either a lipopeptide (or peptide) antibacterial as defined above alone, an aminoglycoside, macrolide or quinolone antimicrobial alone, or a mixture of at least one of the three latter with a lipopeptide (peptide) antimicrobial—in particular with colistin) and the layer of antibacterial agent(s) is further coated with a pH-dependent delivery system (for example an Eudragit polymer layer, more specifically an Eudragit FS30D layer).

Intermediate layers can be provided between the core and the antimicrobial layer, and between the antimicrobial layer and the external layer. This intermediate layer can contain HPMC for example.

In some embodiments, the core is made with an antibacterial such as a peptide antibacterial effective against Gram-negative bacteria—preferably an anti Gram-negative lipopeptide such as a polymyxin, and most preferably colistin—aminoglycoside, macrolide, quinolone or a mixture thereof, obtained following dry or wet granulation techniques. In this embodiment where the core comprises antibacterial(s), it may be necessary to mix said antibacterial(s) with one or more pharmaceutically acceptable carrier as provided below. Granulates of antimicrobial(s) can further be compressed into tablets, using known skills in the art. Subsequent coating of such core is made accordingly to provide delivery of the antibacterial(s) in the late ileum, caecum or colon.

### Additional Formulation Information

The formulations can conveniently be presented in unit dosage form and can be prepared by any methods well known in the art of pharmacy.

Formulations of the invention suitable for oral administration can be in the form of capsules, cachets, pills, tablets, powders, granules, pellets, or as a suspension in an aqueous or non-aqueous liquid, each containing a predetermined amount of an active agent or a combination of active agents.

In solid dosage forms of the invention for oral administration (capsules, tablets, pills, dragees, powders, granules, pellets and the like), the active agent is mixed with one or more pharmaceutically-acceptable carriers, such as (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, starch, (5) wetting agents, such as, for example, cetyl alcohol, glycerol monostearate, and non-ionic surfactants; (6) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions can also comprise buffering agents. Solid compositions of a similar type can also be employed as fillers in soft and hard-shelled gelatin capsules using such excipients as lactose or milk sugars, as well as high molecular weight polyethylene glycols and the like.

A tablet can be made by compression or molding, optionally with one or more accessory ingredients. Compressed tablets can be prepared using binder (for example, gelatin or hydroxypropylmethyl cellulose), lubricant, inert diluent, preservative, disintegrant (for example, sodium starch glyco late or cross-linked sodium carboxymethyl cellulose), surface-active or dispersing agent. Molded tablets can be made by molding in a suitable machine a mixture of the powdered compound moistened with an inert liquid diluent.

The tablets, and other solid dosage forms of the pharmaceutical compositions of the present invention, such as dragees, capsules, pills, granules, can optionally be prepared with coatings and shells, such as gastro-resistant coatings and/or complementary enteric coatings to provide release of the active agent in a certain portion of the gastrointestinal tract and other coatings well known in the pharmaceutical-formulating art.

Examples of embedding compositions which can be used include polymeric substances and waxes. The active agent can also be in micro-encapsulated form, if appropriate, with one or more of the above-described excipients.

The systems with different drug release mechanisms described above can be combined in a final dosage form comprising single or multiple units. Examples of multiple units include multilayer tablets, capsules containing tablets, pellets, granules, etc.

Delayed release formulations are created by coating a solid dosage form with a film of a polymer which is insoluble in the acid environment of the stomach, and soluble in the neutral environment of small intestine.

The delayed release dosage units can be prepared, for example, by coating the delivery system with a selected coating material. The agent-containing composition can be, e.g., a tablet for incorporation into a capsule, a tablet for use as an inner core in a “coated core” dosage form, or a plurality of agent-containing pellets, particles or granules, for incorporation into either a tablet or capsule. Preferred coating materials include bioerodible, gradually hydrolyzable, gradually water-soluble, and/or enzymatically degradable polymers, and can be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon.

Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit®. (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), Eudragits® NE, RL and RS (water-insoluble polymers having different degrees of permeability and expandability) and Eudragit FS30D a tercopolymer of methacrylic acid, methyl acrylate and methylmethacrylate; vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials can also be used. Multi-layer coatings using different polymers can also be applied. The preferred coating weights for particular coating materials can be readily determined by those skilled in the art by evaluating individual release profiles for tablets, pellets and granules prepared with different quantities of various coating materials. It is the combination of materials, method and form of application that produce the desired release characteristics, which one can determine only from the clinical studies.

The coating composition can include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates can also be used. Pigments such as titanium dioxide can also be used. Small quantities of an anti-foaming agent, such as a silicone (e.g., simethicone), can also be added to the coating composition.

Alternatively, a delayed release tablet can be formulated by dispersing the agent within a matrix of a suitable material such as a hydrophilic polymer or a fatty compound. The hydrophilic polymers can be comprised of polymers or copolymers of cellulose, cellulose ester, acrylic acid, methacrylic acid, methyl acrylate, ethyl acrylate, and vinyl or enzymatically degradable polymers or copolymers as described above. These hydrophilic polymers are particularly useful for providing a delayed release matrix. Fatty compounds for use as a matrix material include, but are not limited to, waxes (e.g. carnauba wax) and glycerol tristearate. Once the active agent is mixed with the matrix material, the mixture can be compressed into tablets.

These dosage forms can be administered to humans and other animals for therapy by any suitable route of administration.

Actual dosage levels of the active agents in the pharmaceutical compositions of this invention can be varied so as to obtain an effective removal of any residual antibacterial or chemical or toxin in the intestinal tract, for a particular patient, composition, and mode of administration, without being toxic to the patient.

Particles Incorporating the Active Agents

As mentioned above, modified-release particles including the active agents can be in the form of a sugar-based or microcrystalline cellulose core unit, optionally including a water-soluble coat surrounding the core unit, a drug layer comprising the active agents and a pharmaceutically acceptable binder, and an outer layer for sustained-release and/or controlled-release of the drugs comprising a pH-dependent polymer or copolymer. The “pH dependent” in this context means that the permeability, and accordingly the release characteristics of the drugs can be monitored by the intra-luminal pH, ideally suited to release the contents of the particles in or near the colon. Further layers may additionally be present, although typically either no additional layers are present or an optional outermost film coat layer is additionally present if desired. Further details of the particle components are set forth below.

The core unit of the particles is any core or seed that contains microcrystalline cellulose or sugar and is typically a commercially available microcrystalline cellulose sphere such as Cellets™ or Celpheres™ or Ethispheres™ and Suglets™ for sugar-based pellets for example. The size of the core unit typically has a diameter within the range of 60-2000 microns, preferably the diameter of the core unit is within the range of 700-1000 microns. Generally the core unit constitutes 50-90 wt % of the total weight of the particles composition. In some embodiments, the core unit is 70-90 wt %, while in other embodiments it is preferably 60-70 wt % of the total weight of the particles composition. One benefit of using a unit core is that coating thereof is relatively easy such that hardly any erosion or dissolving of the core unit occurs during coating. Due to this, the coating rate may be increased (e.g., in comparison with sugar cores or “non-pareils”) which reduces the process time and therewith the production costs without adversely affecting the quality of the particles. Also, the thickness of the coat layers on the particles may be easily controlled by the coating and drying time. The calculated yields of the process are more reliable since hardly any core material is lost during the different coating processes.

The core unit can be primarily surrounded by a water soluble coat, such as vinyl pyrrolidone polymer, cellulose polymers or mixtures thereof. If an increased lag time is needed, hydroxylmethyl propyl cellulose (HPMC) may be used in addition to, or in place or, the vinyl pyrrolidone polymer. Typically the water-soluble coat surrounding the core constitutes 2-10 wt % of the total weight of the particles composition.

In one embodiment the pharmaceutical dosage form is prepared with an adequate concentration of both the antimicrobial lipopeptide specific for Gram-negative bacteria, and a macrolide, quinolone or aminoglycoside antibiotic, sprayed onto inert core as described above typically using a binder such as a hydrophilic polymer. Convenient hydrophilic polymer binders include HPMC. Among the commercially available grades, a low viscosity HPMC is generally desired such as Methocel E5™. However, practically any hydrophilic polymer having a sufficient binding property such as PVP, starch, hydrophilic cellulose derivatives, hydrophilic acrylate or methacrylate polymers may be used. The drug layer typically constitutes 2-70 wt % of the total weight of the particles composition. The active agents generally constitute 10-70 wt % of the total weight of the particles.

In another embodiment the pharmaceutical dosage form is prepared with an adequate amount of separately prepared lipopeptide particles and macro lide, quinolone or aminoglycoside particles to match the desired clinical efficacy dose and to be able to prepare adequate dosages according to body weight. Each antibiotic is prepared according to the same scheme as above, i.e., antibiotic solution is sprayed onto inert cores typically using a binder such as a hydrophilic polymer. Convenient hydrophilic polymer binders include hydroxypropyl methyl cellulose (HPMC). Among the commercially available grades, a low viscosity HPMC is generally desired such as Methocel E5™. However, practically any hydrophilic polymer having a sufficient binding property such as PVP, starch, hydrophilic cellulose derivatives, hydrophilic acrylate or methacrylate polymers may be used. The drug layer typically constitutes 2-70 wt % of the total weight of the particles composition. The active agents generally constitute 10-70 wt % of the total weight of the particles.

In a particular embodiment, the outer layer comprises a pH-dependent polymer, especially a Eudragit polymer, as defined above. The amount of polymer in the controlled release layer is typically between 10% and 35%, based on the total weight of the particles. In another embodiment, the outer layer comprises a polymer system which is responsive to time, pressure or bacteria. For example, the outer layer can contain pectin crosslinked with a divalent cation (for example with calcium or zinc, as described above), said pectin being optionally further coated with a cationic polymer (for example with polyethylenimine) or pH dependent (enterosoluble) polymer such as Eudragit, Aqoat or Shellac for examples.

In some embodiments it is advantageous to further include an anti-tacking agent such as talc (which typically constitutes about 10-60 wt %, preferably 25-50%, of the total weight of the layer) and/or glyceryl monostearate (which typically constitutes about 1-5 wt %, preferably 2.5-5%, of the total weight of the layer). Silicon dioxide, such as the SYLOID brand from W.R. Grace, can also be included in the layer. The particles of the invention release the active ingredient preferably in a controlled manner. Alternatively, though it is not generally necessary or desired, the controlled release layer may be combined with a pore forming agent such as HPMC or a plasticizer such as triacetin or a polysorbate to obtain a suitable release profile.

Optionally the particles of the invention may also comprise an outermost film coat for improving the mechanical properties of the particles. Preferably such an outermost film coat is not a functional coat, i.e., it does not substantially modify the controlled release rate of the drug. Such an outermost film coat may include hydroxypropyl methyl cellulose and/or talc, and constitutes about 0.5-2 wt % of the total weight of the particles composition, if present.

The particles can be prepared using any conventional or suitable techniques, and typically involve the following steps: a) providing a core unit with a diameter size specified as described above, b) applying first a water-soluble coat comprising a hydrophilic polymer on said core unit, c) applying secondly the drug layer comprising the lipopeptide antimicrobial agent and the macrolide, quinolone or aminoglycoside antibiotic and a pharmaceutically acceptable binder on the water-soluble coat, d) applying thirdly a controlled-release layer comprising a pH-dependent polymer or copolymer on the drug layer. Optionally a step e) of applying an outermost film coat layer is also carried out. f) combining or not in the final dosage form adequate amount of both particles in a capsule for example, to make the final dosage form.

The coating may be performed in a fluid bed coating equipment, wherein the coats are applied stepwise on the material to be coated. The coating operations are preferably performed by spraying a solution or dispersion of the respective coating materials on the particle to be coated. The liquid carriers of the materials to be coated may be water, a pharmaceutically acceptable organic solvent, such as an aliphatic alcohol (e.g., C₁-C₃ alcohol), or a combination of both. Any method known in the art to apply coats on particles may be used. After any particular coating, the coated material may be dried before applying the next coat.

After the final coating step, the particles are generally cured, usually in the same fluid bed system or in a tray drier system, by heating to a temperature of about 30-80° C. for 1-72 hours. Preferably the curing is performed at a temperature of about 35-50° C. for 2-48 hours, more typically 4-24 hours.

A plurality of modified-release particles can optionally be compressed into a tablet, included into a capsule with appropriate excipients in a manner known in the art to obtain a tablet or a capsule dosage form which contains 50 to 600 mg of each of the active agents. The tablet or capsule, upon dissolution, disintegrates into the separate controlled release particles which individually reach the late ileum or colon where they can release drugs for treating against Gram-negative bacteria.

The type and/or amount of pH-dependent enterosoluble polymer which can be used to coat the core of the invention may be selected by using the Biodis dissolution tester (USP III release apparatus) has provided in the examples. This method may also be used to test other types of drug delivery systems (such as those mentioned above).

The pH-dependent enterosoluble coating can also include various combinations of different pH-dependent enterosoluble polymers. Those skilled in the art are able to select such mixtures of pH-dependent polymers taking into account their general knowledge in this field. For example, a combination of two methacrylic acid polymers such as Eudragit® L100-55 and Eudragit® 5100 can be provided around the core of the invention.

In a particular embodiment, the pH-dependent enterosoluble polymer is selected from
shellac,
anionic copolymers based on methyl acrylate, methyl methacrylate and methacrylic acid, and
methacrylic acid and methyl methacrylate copolymers (1:2 weight ratio).
   In a further particular embodiment, the drug delivery system according the invention comprises:
a core containing an antibacterial such as a lipopeptide (for example a polymyxin, in particular colistin) or another peptide antibacterial effective against Gram-negative bacteria, aminoglycoside, macro lide, quinolone or a mixture thereof (in particular a mixture of a lipopeptide antibacterial (e.g. colistin) with a macrolide, an aminoglycoside or a quinolone antibacterial), and
a layer of an anionic copolymer based on methyl acrylate, methyl methacrylate and methacrylic acid, such as Eudragit® FS30D.
   In another particular embodiment, the drug delivery system according the invention comprises
a core containing an antibacterial such as a lipopeptide (for example a polymyxin, in particular colistin) or another peptide antibacterial effective against Gram-negative bacteria, aminoglycoside, macro lide, quinolone or a mixture thereof (in particular a mixture of a lipopeptide antibacterial with a macrolide, an aminoglycoside or a quinolone antibacterial), and
a layer of shellac.

The external enterosoluble layer may be applied onto the core by any suitable means known to a person skilled in the art. For example, it can be applied using classical fluid bed technology where a water-based or solvent-based solution of coating is applied by spray-drying onto the core pellet. When the weight gain is reached, the formulation can be dried and a further coating can be applied. Multiple coating can thus be applied successively using spray drying technology.

### Intermediate Coating

According to a particular embodiment of the invention, the drug delivery system described above comprises at least one further coating provided between the core described above and the external enteric coating. This further layer(s) (also referred to as “intermediate coating”) is provided to isolate the core composition containing the antibacterial from the external coating. This is particular suitable for sensitive antibacterials such as peptide/lipopeptides or quinolones antibacterials which have incompatibilities with some enteric polymer coatings.

According to a particular embodiment, the intermediate coating is provided onto the core of the invention, and a further coating is applied with a pH-dependent enterosoluble polymer, such as Eudragit™ FS30D (as explained above). The pH-dependent enterosoluble polymer protects the core from the acidic environment found in the upper part of the gastro-intestinal tract. Once the pH-dependent polymer is dissolved, release of the antibacterial can be obtained after the intermediate coating is dissolved.

The intermediate coating can contain pH-dependent or pH-independent polymers.

Among the pH-dependent polymers that can be used as intermediate coating, examples include those described above in “external enterosoluble layer” part, and in particular shellac type polymers such as SSB® Aquagold, anionic copolymers based on methyl acrylate, methyl methacrylate and methacrylic acid such as Eudragit® FS30D, methacrylic acid and ethyl acrylate copolymer such as Eudragit® L30D-55, HPMCAS such as Aqoat AS-MF, MG or HF grades or hydroxypropyl methylcellulose phthalate (HPMCP) such as HP-55 grade.

pH-independent polymers can be selected among slowly water soluble polymers and water insoluble polymers. Non limiting examples of pH-independent water soluble polymers include Polyvinyl pyrolidone (PVP) and high molecular weight cellulose polymers such as hydroxypropylmethylcellulose (HPMC), hydroxypropyl cellulose (HPC). Further non limiting examples of pH-independent insoluble polymers include ethylcellulose polymers and ethyl acrylate methyl methacrylate copolymer (such as Eudragit® NE30D).

In a particular embodiment of the invention, the intermediate coating contains a mixture of polymers. In a first alternative, the mixture of polymers comprises polymers of the same type. For example, the mixture can comprise a pH-dependent polymer with another pH-dependent polymer, a pH-independent soluble polymer with another pH-independent soluble polymer, or a pH-independent insoluble polymer with another pH-independent insoluble polymer. In another alternative, the mixture of polymers comprises polymers of different types. The mixture can comprise a pH-dependent polymer with a pH-independent polymer (either water soluble or insoluble), a pH-independent soluble polymer with a pH-independent insoluble polymer, or a pH-dependent polymer with a pH-independent soluble polymer and a pH-independent insoluble polymer. For example, the intermediate coating can comprise the mixture of a pH-dependent polymer with a pH-independent polymer, such as a mixture of Eudragit® L30D55 with Eudragit® NE30D (for example, in a weight ratio between about 1:9 and about 9:1, in particular between about 2:8 and about 3:7).

The preferred coating and coating component weight ratio can be readily determined by those skilled in the art, for example, by evaluating the release profile of the dosage form, as provided in the examples.

In a particular embodiment, the intermediate coating is selected in order to achieve a delayed and/or controlled release of the antibacterial, as measured by in vitro testing such as BioDis dissolution tester (USP III release apparatus). In this system, the dosage form is successively placed into glass tubes (filled with approx 200 mL of dissolution media) corresponding to the different sections of the gastrointestinal tract, such as described by Fotaki and Coll, in European Journal of Pharmaceutics and Biopharmaceutics 73 (2009) 115-120. This allows a good simulation of in vivo release before testing into mammals. pH, fed state and various other physiological conditions can be tested. Using the BioDis system, it is possible for those skilled in the art to finely tune the formulation to achieve a desired pre-determined delayed release. It will be understood that this method is also useful for testing any kind of drug delivery system intended to deliver the antimicrobial(s) in a desired part of the intestine.

According to the above, a particular embodiment of the invention relates to a drug delivery system comprising:
a core containing an antibacterial such as a lipopeptide (for example a polymyxin, in particular colistin) or other peptide antimicrobials effective against Gram-negative bacteria, aminoglycoside, macro lide, quinolone or a mixture thereof (in particular a mixture of a lipopeptide antibacterial with a macrolide, an aminoglycoside or a quinolone antibacterial),
an external layer of a pH-dependent enterosoluble polymer, and
a coating provided between the core and the external layer.
   In a particular embodiment, the invention relates to a drug delivery system comprising:
a core containing an antibacterial such as a lipopeptide (for example a polymyxin, in particular colistin) or other peptide antimicrobials effective against Gram-negative bacteria, aminoglycoside, macro lide, quinolone or a mixture thereof (in particular a mixture of a lipopeptide antibacterial with a macrolide, an aminoglycoside or a quinolone antibacterial),
a coating selected in the group consisting of HPMC, ethylcellulose and a mixture of methacrylic acid and ethyl acrylate copolymer such as Eudragit® L30D-55 and ethyl acrylate methyl methacrylate copolymer such as Eudragit® NE30D (for example in a mixture ratio of 1:9 to 9:1, preferably of 2:8 to 3:7), and
an external layer of an anionic copolymer based on methyl acrylate, methyl methacrylate and methacrylic acid, such as Eudragit® FS30D.
   In a particular embodiment, the drug delivery system of the invention comprises:
a core containing an antibacterial such as a lipopeptide (for example a polymyxin, in particular colistin) or other peptide antimicrobials effective against Gram-negative bacteria, aminoglycoside, macro lide, quinolone or a mixture thereof (in particular a mixture of a lipopeptide antibacterial with a macrolide, an aminoglycoside or a quinolone antibacterial),
a 15-35% (w/w of the total formulation) coating made of a 2:8 to 3:7 mixture of methacrylic acid and ethyl acrylate copolymer (such as Eudragit® L30D-55) and ethyl acrylate methyl methacrylate copolymer (such as Eudragit® NE30D), and
a 15% (w/w of the total formulation) external layer of an anionic copolymer based on methyl acrylate, methyl methacrylate and methacrylic acid, such as Eudragit® FS30D.
   In a further particular embodiment, the drug delivery system of the invention comprises:
a core containing colistin,
a 15-35% (w/w of the total formulation) coating made of a 2:8 to 3:7 mixture of methacrylic acid and ethyl acrylate copolymer (such as Eudragit® L30D-55) and ethyl acrylate methyl methacrylate copolymer (such as Eudragit® NE30D), and
a 15% (w/w of the total formulation) external layer of an anionic copolymer based on methyl acrylate, methyl methacrylate and methacrylic acid, such as Eudragit® FS30D
The presence of an intermediate coating between colistin and FS30D improves the release of colistin as observed in Bio Diss system.

In another embodiment, the invention relates to a drug delivery system comprising:
an inert core, for example a core made of microcrystalline cellulose (such as Cellets)
a layer of a composition comprising one or more of
   i) a lipopeptide or peptide antibacterial effective against Gram-negative bacteria such as colistin
   ii) an aminoside, macrolide or quinolone antibacterial; and
a system for delivery in the late ileum, caecum or colon, such as an external coating as provided above (for example a pH-dependent polymer or pectin).

An intermediate layer as defined above may be provided between the external layer and the layer containing the antibacterial(s).

In a particular embodiment of the invention, the drug delivery system comprises:
an inert core as defined above;
a layer around said core comprising colistin;
optionally, an intermediate coating around the colistin-containing layer, said intermediate layer being as defined above; and
an external coating for providing delivery in the late ileum, caecum or colon. In this embodiment, the intermediate coating can be selected, for example, in the group consisting of HPMC, ethylcellulose and a mixture of methacrylic acid and ethyl acrylate copolymer such as Eudragit® L30D-55 and ethyl acrylate methyl methacrylate copolymer such as Eudragit® NE30D (for example in a mixture ratio of 1:9 to 9:1, preferably of 2:8 to 3:7).

The invention further relates to the above drug delivery systems comprising colistin for use in all the methods presented herein.

### Methods of Treatment Using the Drug Delivery Systems Described Herein

The compositions described herein can be used to eliminate unwanted Gram-negative bacteria, such as antibiotic-resistant Gram-negative bacteria from the intestine of colonized subjects. As such, the compositions can be used to minimize the increased incidence of ESBL and carbapenemase-producing Gram-negative bacteria.

The active agents can be administered in a therapeutically effective dosage to a patient who has been, or is likely to suffer from, a colonic infection.

The compositions and methods can be used to treat a variety of colonic bacterial infections, or colonization by potentially pathogenic and/or multi-resistant bacteria or yeasts, as well as various disorders resulting from bacterial infections with Gram-negative bacteria such as Escherichia coli and other species of enterobacteria particularly those resistant or multiresistant to antibacterials, Salmonella and shigella species and as well as other potentially pathogenicbacteria such as Pseudomonas aeruginosa, Stenotrophomanas maltophilia, Acinetobacter baumannii and the like. Microbial growth can be inhibited by administering an effective amount of the active agents in a properly designed delivery system and dosage form to a patient in need of such treatment. The exact dose to be administered will vary according to the use of the compositions, age, sex and physiological condition of the patient, and can readily be determined by the treating physician. The compositions may be administered as a single dose or as a multiple dose over a period of time. Doses may be repeated as appropriate.

The compositions can also be used to deliver antibacterial agents to the colon in order to achieve a “selective decontamination,” eliminating commensal and/or potentially pathogenic microorganisms (such as for example enterobacteria, Pseudomonas and other Gram-negative bacteria,) from the colon of patients at risk (such as Intensive care, pre-operative or haemato-oncology patients, for example) before they develop an actual infection.

The compositions can further be used to provide selective decontamination of the colonic bacteria in farm animals, particularly, of specific types of Escherichia coli strains, namely Shiga-toxin Escherichia coli (or STEC), also called Verotoxin Escherichia coli (or VETEC). The use of this method can minimize contamination of the food and water supplies.

The compositions can also be used to eliminate unwanted bacteria in order to help control outbreaks of antibacterial-resistant Gram-negative infections, such as nosocomial infections, in hospitals. Representative infections include nosocomial infections caused by enterobacteria (mostly Klebsiella) resistant to third generation cephalosporins by secretion of an extended spectrum beta-lactamase (ESBL) derived from the TEM or SHV beta-lactamase families, as well as a new type of ESBL, called CTX-M, which have a strikingly different epidemiological pattern of emergence and diffusion. In one embodiment, patients admitted to a hospital, who have been identified positive for one of these bacteria, are selectively decontaminated using specific antibacterial agents targeted to the colon. The same can apply to patients colonized by Gram-negative bacteria that carry other types of antibiotic degrading enzymes such as carbapenemases.

Ideally, the antibacterial active agents delivered to the colon are specific for pathogenic or potentially pathogenic bacteria, and do not have an effect on beneficial bacteria. In this manner, the beneficial bacteria of the commensal flora, are preserved. Accordingly, in one embodiment, the active agents are active against one or more species of harmful bacteria present in the colon, but are either not active, or are less active, against helpful bacteria present in the colon. The combination of the anti-Gram-negative lipopeptide (polymyxin) antibacterial and the macrolide or aminoglycoside antibacterial minimize the problem of bacterial resistance developing against one of these agents.

The present invention will be better understood with reference to the following non-limiting examples.

### Example 1

### Formulation of Azithromycin Dihydrate Pellets for Colonic Delivery with Controlled Drug Delivery Properties

An azithromycin pellet formulation was prepared by spraying an HPC solution of azithromycin onto MCC pellets by the spray drying method using a Mycrolab (Hüttlin GmbH).

| | | Unit formula | Unit formula |
|---|---|---|---|
| Components | Supplier | (g) | (%) |
| CORE COMPOSITION | | | |
| Azithromycin dihydrate | LabExpress | 25 | 16% |
| Cellets 700 | Synthapharm | 100 | 65% |
| HPC-LF (Klucel ™) | Hercules | 5 | 3% |
| Purified water | Qs | 200 | |
| COATING | | | |
| Eudragit FS 30 D | Evonik | 25 | 16% final |
| (35% added weight) | | | |

A solution of azithromycin dihydrate (30 mg/mL) was made and adjusted at pH 7.4, by simple mixing a solution of the drug with an aqueous solution of HPC (7% solution). The solution was then sprayed onto Cellets by spray drying into a MycroLab system. Various coating thicknesses were applied, up to 35% (added weight) FS30D.

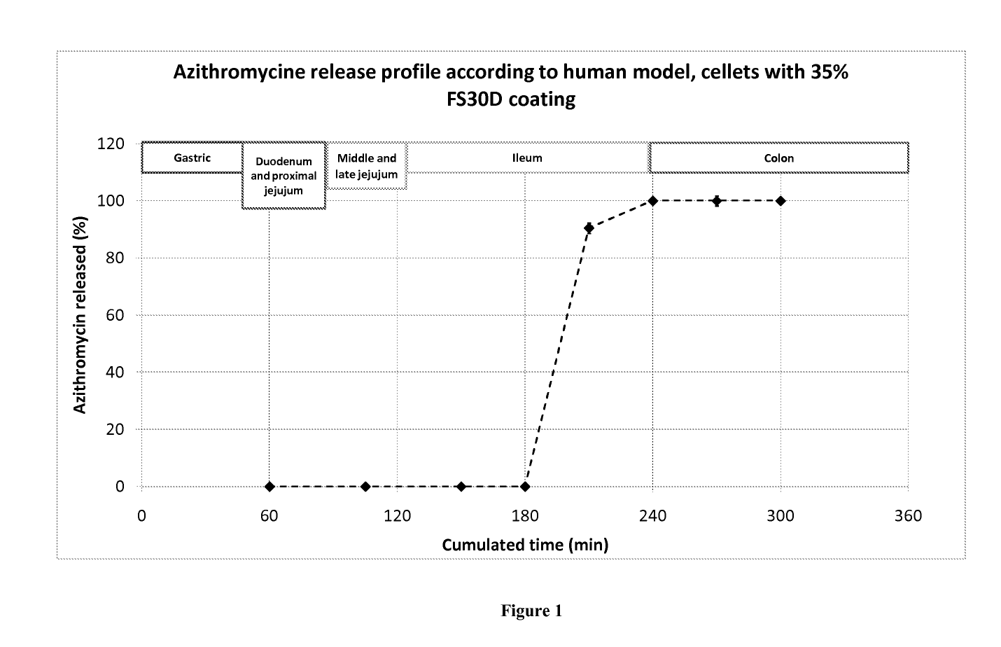

Results of a BioDis assay are presented in FIG. 1 for 35% FS30D coating, but similar results were obtained with 25% FS30D. Legend: FIG. 1: Release of azithromycine in simulated human gastrointestinal tract using a Biodis system. The azithromycin cellet formulation, coated with 35% FS30D (Batch GL-058B4), is successively soaked into simulated fluids such as gastric, proximal jejunum, late jejunum, Ileum and colon. As can be seen on the graph, complete release of azithromycine is reached with FS30D coated cellets.

### Example 2

### Formulation of Colistin Pellets for Colonic Delivery with Controlled Drug Delivery Properties

A colistin pellet formulation was prepared by spraying an HPC solution of colistin sulfate onto MCC pellets by the spray drying method using a Mycrolab (Hüttlin GmbH).

| | | Unit | Unit |
|---|---|---|---|
| Components | Supplier | formula (g) | formula (%) |
| CORE COMPOSITION | | | |
| Colistin sulfate | CEVA | 30 | 16% |
| Cellets 700 | Synthapharm | 100 | 53% |
| HPC-LF (Klucel ™) | Hercules | 5 | 3% |
| Purified water | qs | 200 | |
| PRECOATING | | | |
| Eudragit L30D55/NE30D | Evonik | 27 | 14% final |
| (20% added weight) | | | |
| COATING | | | |
| Eudragit FS 30 D | Evonik | 25 | 13% final |
| (15% added weight) | | | |

A solution of colistin (30 mg/mL) was made and adjusted at pH 7.4, by simple mixing a solution of the drug with an aqueous solution of HPC (7% solution). The solution was then sprayed onto Cellets by spray drying into a MycroLab system.

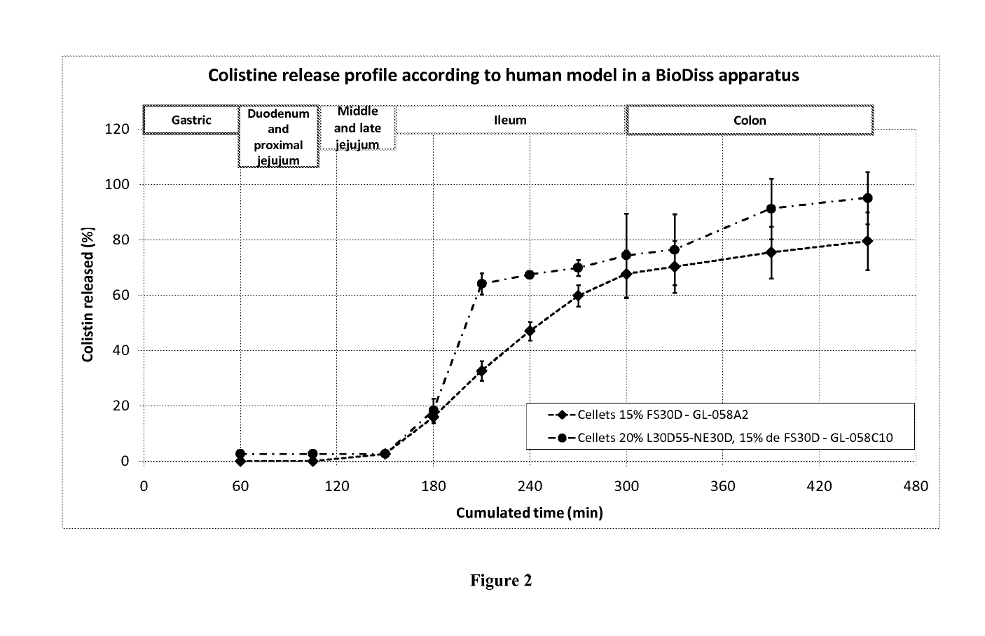

Results are presented in FIG. 2. Legend: Release of colistin in simulated human gastrointestinal tract using a Biodiss system. The colistin cellet formulation, coated with 15% FS30D, with or without pre-coating with 20% L30D55-NE30D is successively soaked into simulated fluids such as gastric, proximal jejunum, late jejunum, Ileum and colon. As can be seen on the graph, complete release in a prolonged manner is reached when an intermediate coating of L30D55-NE30D (3/7 w/w ratio) is provided. In the case of a direct FS30D coating around the colistin layer, complete release is not reached, maybe because of an unexpected interaction of colistin with Eudragit FS30D. Thus, it may be preferred to separate the colistin layer and the Eudragit FS30D layer to reduce to the doses of colistin which may be necessary to obtain the desired decontamination.

### Example 3

### Coatings of Various Formulation of Colistin Pellets for Colonic Delivery with Controlled Drug Delivery Properties

Colistin pellet formulations were prepared using various coatings thickness and compositions. In the case of colistin, an incompatibility has been observed with the external coating made of FS30D that prevent from total colistin release as measured in BioDis system in vitro. To circumvent this, we tested various subcoats compositions.

All formulations were prepared using the protocol of example 2.

| | | | Coating | |
|---|---|---|---|---|
| | | | (added | |
| | | | weight | |
| | | | to the core | |
| Batch | Active | | composition) | Colistin |
| number | drug | Coating details | (w/w) | (μg/mg |
| GL-058 C1 | colistin | HPMC HMW | 15% | 217 |
| GL-058 C2 | colistin | HPMC | 8% | 202 |
| GL-058 C3 | colistin | HPMC-L30D55 | 15% | 180 |
| GL-058 C4 | colistin | HPMC-L30D55 | 20% | 172 |
| GL-058 C5 | colistin | HPMC-L30D55 | 25% | 166 |
| GL-058 C5 | colistin | HPMC-L30D55 | 25% | 166 |
| GL-058 C6 | colistin | HPMC-L30D55/NE30D | 15% | 177 |
| GL-058 C7 | colistin | HPMC-L30D55/NE30D | 20% | 169 |
| GL-058 C8 | colistin | HPMC-L30D55/NE30D | 25% | 162 |
| GL-058 C9 | colistin | L30D55/NE30D | 20% | 166 |
| GL-058 C10 | colistin | L30D55/NE30D + | 20% and | 144 |
| | | FS30D | 15% | |

### Example 4

### Formulation of Colistin Pellets for Colonic Delivery

A colistin formulation is prepared via wet granulation followed by extrusion spheronization process using a Bosch Profimix 44 granulator and a Nica Spheronizer S-450.

Colistin is mixed with Gelcarin GP 911 from FMC Biopolymers and wet granulated with sufficient purified water to obtain a mix which is then extruded (the feeder is set to 50 rpm and the impeller to 70 rpm) and spheronised to obtain pellets of approximately 1 to 3 mm in diameter (speed 750 rpm).

The pellets obtained after the spheronization step are unified and are transferred into the bowl of the fluid bed dryer. Pellets are dried for 40 minutes at a temperature of 70° C. (inlet air)

Pellets are then transferred to the MP-1 FlexStream and coated with Eudragit™ type polymers.

| | | Unit formula | Unit formula |
|---|---|---|---|
| Components | Supplier | (g) | (%) |
| CORE COMPOSITION | | | |
| Colistin sulfate | CEVA | 50 | 60% |
| Gelcarin GP911 | Shin Etsu | 10 | 12% |
| Purified water | qs | | |
| PRECOATING | | | |
| Eudragit L30D55/NE30D | Evonik | 12 | 14% final |
| (20% added weight) | | | |
| COATING | | | |
| Eudragit FS 30 D | Evonik | 11 | 13% final |
| (15% added weight) | | | |

The same formulation can be prepared using other excipients including microcrystalline cellulose (MCC), starch, lactose, dicalcium phosphate (DCP), and sugar.

Substitution of a colistin salt is possible, and similar formulations can be prepared using Colistin sodium methanesulfonate from Bacillus colistinus.

### Example 5

### Formulation of Colistin Sulfate Pellets for Colonic Delivery

A colistin sulfate formulation is prepared via wet granulation followed by extrusion spheronization process using a Bosch Profimix 44 granulator and a Nica Spheronizer S-450.

Colistin is mixed with Gelcarin GP 911 and Avicel PH101 from FMC Biopolymers and wet granulated with sufficient purified water to obtain a mixture, which is then extruded (the feeder is set to 50 rpm and the impeller to 70 rpm) and spheronized to obtain pellets of approximately 1 to 3 mm in diameter (speed 750 rpm).

The pellets obtained after the spheronization step are unified and are transferred into the bowl of the fluid bed dryer. Pellets are dried for 40 minutes at a temperature of 70° C. (inlet air)

Pellets are then transferred to the MP-1 FlexStream and coated with Eudragit™ type polymers.

| | | Unit formula | Unit formula |
|---|---|---|---|
| Components | Supplier | (g) | (%) |
| CORE COMPOSITION | | | |
| Colistin sulfate | CEVA | 25 | 36% |
| Gelcarin GP911 | Shin Etsu | 7 | 10% |
| Avicel PH102 | FMC | 18 | 26% |
| Purified water | qs | | |
| PRECOATING | | | |
| Eudragit L30D55/NE30D | Evonik | 10 | 14% final |
| (20% added weight) | | | |
| COATING | | | |
| Eudragit FS 30 D | Evonik | 9 | 13% final |
| (15% added weight) | | | |

Alternatively, the granulation mixture can also serve for making tablets. The same colistin formulation is prepared via wet granulation in a Bosch Profimix 44 granulator followed by tabletting. Colistin is mixed with Gelcarin GP 911 and Avicel PH102 from FMC Biopolymers and wet granulated with sufficient purified water to obtain a mix which is dried and subsequently compressed into tablets.

The tablets obtained are coated with Eudragit™ type polymers in a classical fluid bed apparatus.

### Example 6

### Formulation of Azithromycin Dihydrate Pellets for Colonic Delivery

An azithromycin dihydrate formulation is prepared via wet granulation followed by extrusion spheronization process using a Bosch Profimix 44 granulator and a Nica Spheronizer S-450.

Azithromycin is mixed with Gelcarin GP 911 and Avicel PH101 from FMC Biopolymers and wet granulated with sufficient purified water to obtain a mix which is then extruded (the feeder is set to 50 rpm and the impeller to 70 rpm) and spheronized to obtain pellets of approximately 1 to 3 mm in diameter (speed 750 rpm).

The pellets obtained after the spheronization step are unified and are transferred into the bowl of the fluid bed dryer. Pellets are dried for 40 minutes at a temperature of 70° C. (inlet air)

Pellets are then transferred to the MP-1 FlexStream and coated with Eudragit™ type polymers.

| | | Unit formula | Unit formula |
|---|---|---|---|
| Components | Supplier | (g) | (%) |
| CORE COMPOSITION | | | |
| Azithromycin dihydrate | LabExpress | 25 g | 38% |
| Gelcarin GP911 | Shin Etsu | 5 g | 8% |
| Avicel PH101 | FMC | 20 g | 30% |
| Purified water | qs | | |
| COATING | | | |
| Eudragit FS 30 D | Evonik | 7 g | 11% final |
| (15% added weight) | | | |

Alternatively, the granulation mixture can also serve for making tablets.

The same azithromycin dihydrate formulation is prepared via wet granulation in a Bosch Profimix 44 granulator followed by tabletting. Azithromycin is mixed with Gelcarin GP 911 and Avicel PH102 from FMC Biopolymers and wet granulated with sufficient purified water to obtain a mix which is dried and subsequently compressed into tablets.

The tablets obtained are coated with Eudragit™ type polymers in a classical fluid bed apparatus.

### Example 7

### Formulation of Combined Azithromycin Dihydrate and Colistin Pellets for Colonic Delivery with Controlled Drug Delivery Properties

A combined azithromycin dihydrate and colistin pellet formulation is prepared by spraying an HPMC/HPC solution of both antibiotics onto MCC pellets by the spray drying method using a Mycrolab (Hüttlin GmbH).

| | | Unit formula | Unit formula |
|---|---|---|---|
| Components | Supplier | (g) | (%) |
| CORE COMPOSITION | | | |
| Azithromycin dihydrate | LabExpress | 25 g | 7% |
| Colistin sulfate | Molekula | 25 g | 7% |
| Cellets 700 | Synthapharm | 200 g | 55% |
| HPMC 606 | Shin Etsu | 12 g | 3% |
| HPC-LF (Klucel ™) | Hercules | 2 g | 1% |
| Purified water | qs | | |
| PRECOATING | | | |
| Eudragit L30D55/NE30D | Evonik | 52 g | 14% final |
| (20% added weight) | | | |
| COATING | | | |
| Eudragit FS 30 D | Evonik | 46 g | 13% final |
| (15% added weight) | | | |

A solution of azithromycin dihydrate (30 mg/mL) and colistin is made and adjusted at pH 7.4, by simple mixing a solution of the drugs with an aqueous solution of HPMC and HPC (7% solution). The solution is then sprayed onto Cellets by spray drying into a MycroLab system.

### Example 8

### Formulation of Combined Azithromycin Dihydrate and Colistin Pellets for Colonic Delivery with Controlled Drug Delivery Properties

A combined formulation of antibiotics can be prepared by simple mixing of both colonic-delivery formulations as described above in a capsule dosage form.

Adequate dosage of each antibiotic can be easily controlled on a weight by weight basis to provide a final dosage form with clinically relevant doses of each antibiotic.

### Example 9

### Formulation of Neomycin Pellets for Colonic Delivery

A neomycin formulation is prepared via wet granulation followed by extrusion spheronization process using a Bosch Profimix 44 granulator and a Nica Spheronizer S-450.

Neomycin is mixed with Gelcarin GP 911 from FMC Biopolymers and wet granulated with sufficient purified water to obtain a mix which is then extruded (the feeder is set to 50 rpm and the impeller to 70 rpm) and spheronised to obtain pellets of approximately 1 to 3 mm in diameter (speed 750 rpm).

The pellets obtained after the spheronization step are unified and are transferred into the bowl of the fluid bed dryer. Pellets are dried for 40 minutes at a temperature of 70° C. (inlet air)

Pellets are then transferred to the MP-1 FlexStream and coated with Eudragit™ type polymers.

| | | Unit formula | Unit formula |
|---|---|---|---|
| Components | Supplier | (g) | (%) |
| CORE COMPOSITION | | | |
| Neomycin sulfate | Molekula | 50 g | 44% |
| Gelcarin GP911 | Shin Etsu | 9 g | 8% |
| Purified water | qs | | |
| COATING | | | |
| Eudragit FS 30 D | Evonik | 9 g | 8% |
| (15% added weight) | | | |

The same formulation can be prepared using other excipients including microcrystalline cellulose (MCC), starch, lactose, dicalcium phosphate (DCP), and sugar.

### Example 10

### Formulation of Gentamycin Pellets for Colonic Delivery

A gentamycin formulation is prepared via wet granulation followed by extrusion spheronization process using a Bosch Profimix 44 granulator and a Nica Spheronizer S-450.

Gentamycin is mixed with Gelcarin GP 911 from FMC Biopolymers and wet granulated with sufficient purified water to obtain a mix which is then extruded (the feeder is set to 50 rpm and the impeller to 70 rpm) and spheronised to obtain pellets of approximately 1 to 3 mm in diameter (speed 750 rpm).

The pellets obtained after the spheronization step are unified and are transferred into the bowl of the fluid bed dryer. Pellets are dried for 40 minutes at a temperature of 70° C. (inlet air)

Pellets are then transferred to the MP-1 FlexStream and coated with Eudragit™ type polymers.

| | | Unit formula | Unit formula |
|---|---|---|---|
| Components | Supplier | (g) | (%) |
| CORE COMPOSITION | | | |
| Gentamycin sulfate | Molekula | 50 g | 44% |
| Gelcarin GP911 | Shin Etsu | 9 g | 8% |
| Purified water | qs | | |
| PRECOATING | | | |
| Eudragit L30D55/NE30D | Evonik | 52 g | 14% final |
| (20% added weight) | | | |
| COATING | | | |
| Eudragit FS 30 D | Evonik | 46 g | 13% final |
| (15% added weight) | | | |

The same formulation can be prepared using other excipients including microcrystalline cellulose (MCC), starch, lactose, dicalcium phosphate (DCP), and sugar.

### Example 11

### Formulation of Spectinomycin Pellets for Colonic Delivery

A spectinomycin formulation is prepared via wet granulation followed by extrusion spheronization process using a Bosch Profimix 44 granulator and a Nica Spheronizer S-450.

Spectinomycin is mixed with Gelcarin GP 911 from FMC Biopolymers and wet granulated with sufficient purified water to obtain a mix which is then extruded (the feeder is set to 50 rpm and the impeller to 70 rpm) and spheronised to obtain pellets of approximately 1 to 3 mm in diameter (speed 750 rpm).

The pellets obtained after the spheronization step are unified and are transferred into the bowl of the fluid bed dryer. Pellets are dried for 40 minutes at a temperature of 70° C. (inlet air)

Pellets are then transferred to the MP-1 FlexStream and coated with Eudragit™ type polymers.

| | | Unit formula | Unit formula |
|---|---|---|---|
| Components | Supplier | (g) | (%) |
| CORE COMPOSITION | | | |
| Spectinomycin sulfate | Molekula | 50 g | 44% |
| Gelcarin GP911 | Shin Etsu | 9 g | 8% |
| Purified water | qs | | |
| COATING | | | |
| Eudragit FS 30 D | Evonik | 9 g | 8% |
| (15% added weight) | | | |

The same formulation can be prepared using other excipients including microcrystalline cellulose (MCC), starch, lactose, dicalcium phosphate (DCP), and sugar.

### Example 12

### Formulation of Combined Aminoside and Polymyxin Pellets for Colonic Delivery with Controlled Drug Delivery Properties

A combined amino side and polymyxin pellet formulation is prepared by spraying an HPMC/HPC solution of both antibiotics onto MCC pellets by the spray drying method using a Mycrolab (Hüttlin GmbH).

| | | Unit | Unit |
|---|---|---|---|
| | | formula | formula |
| Components | Supplier | (g) | (%) |
| GRANULATION | | | |
| Aminoside (gentamycin, | | 25 g | 7% |
| neomycin or spectinomycin for | | | |
| example) | | | |
| Polymyxin (colistin for example) | | 25 g | 7% |
| Cellets 700 | Synthapharm | 200 g | 55% |
| HPMC 606 | Shin Etsu | 12 g | 3% |
| HPC-LF (Klucel ™) | Hercules | 2 g | 1% |
| Purified water | qs | | |
| PRECOATING | | | |
| Eudragit L30D55/NE30D | Evonik | 52 g | 14% final |
| (20% added weight) | | | |
| COATING | | | |
| Eudragit FS 30 D | Evonik | 46 g | 13% final |
| (15% added weight) | | | |

A solution of amino side (30 mg/mL) and colistin is made and adjusted at pH 7.4, by simple mixing a solution of the drugs with an aqueous solution of HPMC and HPC (7% solution). The solution is then sprayed onto Cellets by spray drying into a MycroLab system.

### Example 13

### Formulation of Combined Aminoside and Colistin Pellets for Colonic Delivery with Controlled Drug Delivery Properties

A combined formulation of antibiotics can also be prepared by simple mixing of both colonic-delivery formulations in the form of pellets for example as described above in a capsule dosage or in tablet form.

Adequate dosage of each antibiotic can be easily controlled on a weight by weight basis to provide a final dosage form with clinically relevant doses of each antibiotic.

### Example 14

### Formulation of Spectinomycin Pellets or Tablets for Colonic Delivery

Spectinomycin is mixed with Gelcarin GP 911 and Avicel PH101 from FMC Biopolymers and wet granulated with sufficient purified water to obtain a mixture, which can then be extruded using a Bosch Profimix 44 granulator and a Nica Spheronizer S-450 (the feeder is set to 50 rpm and the impeller to 70 rpm) and spheronized to obtain pellets of approximately 1 to 3 mm in diameter (speed 750 rpm) or tableted using tableting machine.

The pellets obtained after the spheronization step are unified and are transferred into the bowl of the fluid bed dryer. Pellets are dried for 40 minutes at a temperature of 70° C. (inlet air)

Pellets are then transferred to the MP-1 FlexStream and coated with Eudragit™ type polymers.

The tablets obtained are coated with Eudragit™ type polymers in a classical fluid bed apparatus.

| | | Unit formula | Unit formula |
|---|---|---|---|
| Components | Supplier | (g) | (%) |
| CORE COMPOSITION | | | |
| Spectinomycin | Molekula | 80 | 58% |
| Gelcarin GP911 | Shin Etsu | 10 | 7% |
| Avicel PH102 | FMC | 10 | 7% |
| Purified water | qs | | |
| PRECOATING | | | |
| Eudragit L30D55/NE30D | Evonik | 20 | 14% final |
| (20% added weight) | | | |
| COATING | | | |
| Eudragit FS 30 D | Evonik | 18 | 14% final |
| (15% added weight) | | | |

### Example 15

### In Vitro Assessment of the Efficacy of the Decontaminating Combinations on Target Bacteria and on the Rest of the Microflora

To assess the efficacy of the product, the minimum inhibitory concentrations (MICs) of each of the decontaminating agents is tested on the target bacteria by assessing their growth on solid media containing increasing concentrations of either of the agents. The MIC is defined as the concentration on which no visible growth is observed after 24 hrs of incubation at 37° C.

Median MIC of each decontaminating agent on a representative sample made of 100 target bacteria is calculated, as well as MIC50 and MIC 90 (these are defined as MICs that inhibit the growth of 50% or 90%, respectively, of the target strains from the representative sample).
Target bacteria are defined as multiresistant Gram-negative bacteria such as those that produce extended spectrum beta-lactamases and those that produce carbapenemases. For example, ESBL resistant CTX-M, TEM-derived, SHV-derived producing strains and carbapenemase-producing strains such as Klebsiella pneumoniae carbapenemase (KPC), VIM-, GES-producing strains are tested according to this method.
MICs of each of the decontaminating agents on the target bacteria are compared to the concentrations of these agents that are obtained in the lower part of the intestinal tract (late ileum, caecum and colon) of the decontaminated subjects. The ratio of the median MIC, MIC50 and MIC90 to the concentrations of the decontaminating agent in the feces is calculated. These ratios are referred to below as efficacy ratio (ER).
Using the same methodology, the ER of the agents on other bacteria from the colonic flora, which are not the ones that are targeted by the decontaminating combination, are assessed. The other bacteria include commensal members of the Bacteroides and Clostridia families which are known to be protective of the integrity of the colonic flora.
The combination of decontaminating agents that is preferably the one that associates the agents which have (i) the lowest ER on the target bacteria, and (ii) the highest ER on other bacteria.

All documents cited above are hereby incorporated in their entirety by reference. From the foregoing, it will be obvious to those skilled in the art that the present embodiments and examples are to be considered in all respects as illustrative and not restrictive, and all changes which come within the meaning and range of equivalency of the claims are therefore intended to be embraced therein. All documents referred to herein are hereby incorporated by reference.

1. A composition comprising a drug delivery system that is orally administered, and delivers specifically to the late ileum, caecum, or colon, and substantially avoids delivery to other areas of the gastrointestinal tract, wherein the drug delivery system comprises:
a) an aminoglycoside, quinolone or macrolide antibacterial, and
b) an anti-Gram-negative lipopeptide antibacterial or other peptide antibacterial effective against Gram-negative bacteria.

2. The composition of claim 1, wherein the lipopeptide antibacterial is colistin.

3. The composition claim 1, wherein the drug delivery system comprises an aminoglycoside antibacterial selected in the group consisting of spectinomycin, gentamicin, amikacin, arbekacin, kanamycin, neomycin, netilmicin, paromomycin, rhodostreptomycin, streptomycin, tobramycin and apramycin.

4. The composition of claim 1, wherein the macrolide is azithromycin.

5. A set of a first and a second compositions, wherein
the first composition comprises an aminoglycoside, macrolide or quinolone antibacterial, and
the second composition comprises a drug delivery system that is orally administered, and delivers specifically to the late ileum, caecum, or colon, and substantially avoids delivery to other areas of the gastrointestinal tract, wherein the drug delivery system comprises an anti-Gram-negative lipopeptide antibacterial or other peptide antibacterial effective against Gram-negative bacteria.

6. The set according to claim 5, wherein the first composition is a drug delivery system that is orally administered, and delivers specifically to the late ileum, caecum, or colon, and substantially avoids delivery to other areas of the gastrointestinal tract, wherein the drug delivery system comprises an aminoglycoside, macrolide or quinolone antibacterial.

7. A method for providing elimination of Gram-negative resistant bacteria from the colon of a patient colonized by such bacteria, comprising administering the composition of claim 1 to a patient in need of treatment thereof.

8. The method of claim 7, wherein the method provides elimination of Gram-negative resistant bacteria from the colon of a patient at risk before such patient develops an actual infection.

9. The method according to claim 7, wherein the method provides elimination of pathogenic microbes within the lumen of the intestinal tract, and minimizes the pathogenic alterations of the mucosa resulting from the action of compounds released by infecting bacteria.

10. The method according to claim 7, wherein the method provides elimination of Gram-negative bacteria from the colon of farm animals.

11. The method of claim 10, wherein the colonic bacteria to be targeted are Shiga-toxin Escherichia coli.

12. The method according to claim 7, wherein the method provides selective decontamination in a patient to control outbreaks of antibacterial-resistant Gram-negative infections in hospitals.

13. The method of claim 12, wherein the nosocomial infection is caused by a) Gram-negative bacteria which are resistant to third generation cephalosporins by secretion of an extended spectrum beta-lactamase derived from the TEM or SHV beta-lactamase families, b) Gram-negative bacteria which are resistant to third generation cephalosporins by secretion of an extended spectrum beta-lactamase derived from CTX-M beta-lactamase family, or c) Gram-negative bacteria which are resistant to antibacterials by secretion of other types of enzymes.

14. The method according to claim 7, wherein the method reduces the concentration of bacteria in the colon of a patient who has a colonic bacterial infection, or who is at risk of having a colonic bacterial infection.

15. The method of claim 14, wherein the composition further comprises a third active agent, where the third active agent is an anti-inflammatory compound, an anti-histamine, an anti-cholinergic, an antiviral, an antimitotic, a diagnostic agent, or an immunosuppressive agent.

16. A kit comprising:
a first composition comprising a drug delivery system that is orally administered, and delivers specifically to the late ileum, caecum, or colon, and substantially avoids delivery to other areas of the gastrointestinal tract, wherein the drug delivery system comprises an anti-Gram-negative lipopeptide antibacterial or other peptide antibacterial effective against Gram-negative bacteria, and
a second composition comprising an aminoglycoside, macrolide or quinolone antibacterial.

17. A method for providing elimination of Gram-negative resistant bacteria from the colon of a patient colonized by such bacteria, comprising administering a set of compositions according to claim 5 to a patient in need of treatment thereof.

18. The method according to claim 17, wherein the method provides elimination of Gram-negative resistant bacteria from the colon of a patient at risk before such patient develops an actual infection.

19. The method of claim 17, wherein the method provides elimination of pathogenic microbes within the lumen of the intestinal tract, and minimizes the pathogenic alterations of the mucosa resulting from the action of compounds released by infecting bacteria.

20. The method of claim 17, wherein the method provides elimination of Gram-negative bacteria from the colon of farm animals.

21. The method of claim 20, wherein the colonic bacteria to be targeted are Shiga-toxin Escherichia coli.

22. The method of claim 17, wherein the method provides selective decontamination in a patient to control outbreaks of antibacterial-resistant Gram-negative infections in hospitals.

23. The method of claim 22, wherein the nosocomial infection is caused by a) Gram-negative bacteria which are resistant to third generation cephalosporins by secretion of an extended spectrum beta-lactamase derived from the TEM or SHV beta-lactamase families, b) Gram-negative bacteria which are resistant to third generation cephalosporins by secretion of an extended spectrum beta-lactamase derived from CTX-M beta-lactamase family, or c) Gram-negative bacteria which are resistant to antibacterials by secretion of other types of enzymes.

24. The method of claim 17, wherein the method reduces the concentration of bacteria in the colon of a patient who has a colonic bacterial infection, or who is at risk of having a colonic bacterial infection.

25. The method of claim 24, wherein the composition further comprises a third active agent, where the third active agent is an anti-inflammatory compound, an anti-histamine, an anti-cholinergic, an antiviral, an antimitotic, a diagnostic agent, or an immunosuppressive agent.
